SEQUENCE LISTING (25 sequences)

SEQ ID NO: 1 - DNA, 2238 bp, Artificial Sequence
  Synthetic construct
atggcgaacaagcacctgagccttagcctcttcctcgtgctcctgggcctctccgcctccctcgcctccggcgtcacaaggtgctcggctaacaccaacttccgcaggttcttctccaagttcaacgtgcaggctcccgccgtggtggtgctcggcggctacctgccaatcggcgagaaccagggcgtgaattcaacgtggtactgcgcgggccagcaccccacggcctcgggcgtccacggcatcttcgtgagccacatccgcggcggccacggcttcgagatcggcatctcgcaggagcccttcgacccatcgggctaccagctctacctgcacaaggccacgaacggcaacacgaacgctaccgccaggctgaggatctgccagttcccctccatcaagaccctgggcccaaccgcgaacaacgacgtgaccacgggcaggaactgcctgttcaacaaggccatcccggcgcacatgtccgagcacagcgtcgtgggcatcacgtgggacaacgaccgcgtcaccgtgttcagcgacaagatctactacttctacttcaagaacgactggtccagggtcgccaccaagtgctacaacagcggcggctgcgcgatgcagtatgtgtacgagccgacctactacatgctgaacgtgacgtcggcgggcgaggacggcatcagctaccagccctgcacggccaactgcatcggctacgccgcgaacgtgttcgccacggagcccaacggccacatcccagagggcttctccttcaacaactggttcctcctgtccaacgacagcaccctggtccacggcaaggtcgtgagcaaccagccgctcctggtgaactgcctcctggcgatccccaagatctacggcctcggccagttcttctccttcaaccagacgatcgacggcgtctgcaacggcgcggccgtgcagagggctcccgaggcgctgaggttcaacatcaacgacacctccgtcatcctggcggagggcagcatcgtgctccacacggccctgggcacgaacttctccttcgtgtgcagcaactccagcaacccccacctcgccacgttcgccatccccctgggcgctacccaggtcccctactactgcttcctgaaggtggacacctacaacagcaccgtctacaagttcctggccgtgctgccgcccacggtccgcgagatcgtgatcaccaagtacggcgacgtctacgtgaacggcttcggctacctccacctgggcctcctggacgcggtcaccatcaacttcaccggccacggcacggacgacgacgtgtccggcttctggacgatcgccagcaccaacttcgtggacgccctgatcgaggtgcagggcaccgccatccagcgcatcctctactgcgacgacccggtgtcccagctgaagtgcagccaggtggcgttcgacctcgacgacggcttctaccccatctccagcaggaacctcctgtcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgagcttcatgaccctggacgtgtgcaccaagtacacgatctacggcttcaagggcgagggcatcatcaccctcacgaactcaagctttctggccggcgtctactacacctccgacagcggccagctcctggccttcaagaacgtgacctcgggcgccgtctactcggtgacgccctgctccttcagcgagcaggctgcctacgtggacgacgacatcgtcggcgtgatctccagcctctcctcttcgactttcaactcaacccgcgagctgcccggcttcttctaccattccaaggacgagctctgatag

SEQ ID NO: 2 - DNA, 2415 bp, Artificial Sequence
  Synthetic construct
atggcgaacaagcacctgagccttagcctcttcctcgtgctcctgggcctctccgcctccctcgcctccggcgtcacaaggtgctcggctaacaccaacttccgcaggttcttctccaagttcaacgtgcaggctcccgccgtggtggtgctcggcggctacctgccaatcggcgagaaccagggcgtgaattcaacgtggtactgcgcgggccagcaccccacggcctcgggcgtccacggcatcttcgtgagccacatccgcggcggccacggcttcgagatcggcatctcgcaggagcccttcgacccatcgggctaccagctctacctgcacaaggccacgaacggcaacacgaacgctaccgccaggctgaggatctgccagttcccctccatcaagaccctgggcccaaccgcgaacaacgacgtgaccacgggcaggaactgcctgttcaacaaggccatcccggcgcacatgtccgagcacagcgtcgtgggcatcacgtgggacaacgaccgcgtcaccgtgttcagcgacaagatctactacttctacttcaagaacgactggtccagggtcgccaccaagtgctacaacagcggcggctgcgcgatgcagtatgtgtacgagccgacctactacatgctgaacgtgacgtcggcgggcgaggacggcatcagctaccagccctgcacggccaactgcatcggctacgccgcgaacgtgttcgccacggagcccaacggccacatcccagagggcttctccttcaacaactggttcctcctgtccaacgacagcaccctggtccacggcaaggtcgtgagcaaccagccgctcctggtgaactgcctcctggcgatccccaagatctacggcctcggccagttcttctccttcaaccagacgatcgacggcgtctgcaacggcgcggccgtgcagagggctcccgaggcgctgaggttcaacatcaacgacacctccgtcatcctggcggagggcagcatcgtgctccacacggccctgggcacgaacttctccttcgtgtgcagcaactccagcaacccccacctcgccacgttcgccatccccctgggcgctacccaggtcccctactactgcttcctgaaggtggacacctacaacagcaccgtctacaagttcctggccgtgctgccgcccacggtccgcgagatcgtgatcaccaagtacggcgacgtctacgtgaacggcttcggctacctccacctgggcctcctggacgcggtcaccatcaacttcaccggccacggcacggacgacgacgtgtccggcttctggacgatcgccagcaccaacttcgtggacgccctgatcgaggtgcagggcaccgccatccagcgcatcctctactgcgacgacccggtgtcccagctgaagtgcagccaggtggcgttcgacctcgacgacggcttctaccccatctccagcaggaacctcctgtcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgagcttcatgaccctggacgtgtgcaccaagtacacgatctacggcttcaagggcgagggcatcatcaccctcacgaactcaagctttctggccggcgtctactacacctccgacagcggccagctcctggccttcaagaacgtgacctcgggcgccgtctactcggtgacgccctgctccttcagcgagcaggctgcctacgtggacgacgacatcgtcggcgtgatctccagcctctcctcttcgactttcaactcaacccgcgagctgcccggcttcttctaccattccaacgatggctccaattgcacagagcctgtgttggtgtacagcaacatcggtgtttgcaaatctggcagcattggctacgtcccatcccagtctggccaagtgaagattgccccgaccgttactgggaacatcagcattcccaccaacttcagcttctacccctcctaccacagcaccccacagcgcccctgatag

SEQ ID NO: 3 - protein, 716 aa, Porcine epidemic diarrhea virus
VTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHS

SEQ ID NO: 4 - protein, 767 aa, Porcine epidemic diarrhea virus
VTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFS

SEQ ID NO: 5 - DNA, 72 bp, Hordeum vulgare
atggcgaacaagcacctgagccttagcctcttcctcgtgctcctgggcctctccgcctccctcgcctccggc

SEQ ID NO: 6 - protein, 24 aa, Hordeum vulgare
MANKHLSLSLFLVLLGLSASLASG

SEQ ID NO: 7 - DNA, 756 bp, Artificial Sequence
  Synthetic construct
atggcgaacaagcacctgagccttagcctcttcctcgtgctcctgggcctctccgcctccctcgcctccggcgtgtccaacggctcaatcccagtggacgaggtcatccagcacctccgcaactggaacttcacgtggaacatcatcctcaccatcctcctggtggtcctgcagtacggccactacaagtactccgccttcctgtacggcgtcaagatggcgatcctctggatcctctggcccctggtgctcgctctctccctgttcgacgcttgggccagcttccaggtgaactgggtcttcttcgccttctccatcctcatggcgtgcatcaccctcatgctgtggatcatgtacttcgtcaacagcatcaggctgtggaggaggacccactcctggtggagcttcaaccccgagacggacgctctcctgaccacctccgtcatgggcaggcaggtgtgcatccccgtcctgggcgccccaaccggcgtgaccctcacgctgctgtcgggcaccctcctggtggagggctacaaggtggctaccggcgtgcaggtcagccagctccccaacttcgtgaccgtcgccaaggcgaccacgacgatcgtgtacggcagggtgggcaggtcggtgaacgcttcctcgggcaccggctgggcgttctacgtgaggtccaagcacggcgactacagcgcggtgagcaacccctcgtcggtcctgactgattcggagaaggtcctgcacctcgtgtagtga

SEQ ID NO: 8 - protein, 250 aa, Artificial Sequence
  Synthetic construct
MANKHLSLSLFLVLLGLSASLASGVSNGSIPVDEVIQHLRNWNFTWNIILTILLVVLQYGHYKYSAFLYGVKMAILWILWPLVLALSLFDAWASFQVNWVFFAFSILMACITLMLWIMYFVNSIRLWRRTHSWWSFNPETDALLTTSVMGRQVCIPVLGAPTGVTLTLLSGTLLVEGYKVATGVQVSQLPNFVTVAKATTTIVYGRVGRSVNASSGTGWAFYVRSKHGDYSAVSNPSSVLTDSEKVLHLV

SEQ ID NO: 9 - protein, 713 aa, Porcine epidemic diarrhea virus
VTRCQSTINFRRFFSKFNVQAPAVVVLGGYLPSMNSSSWYCGTGIETDSGVHGIFLSYIDSGQGFEIGISQEPFDPSGYQLYLHKATNGNTNAIARLRICQFPDNKTLGPTVNDVTTGRNCLFNKAIPAYLQDGKNIVVGITWDNDRVTVFADKIYHFYLKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLLAIPKIYGLGQFFSFNQTMDGVCNGAAAQRAPEALRFNINDTSVILAEGSIVLHTALGTNLSFVCSNSSDPHLAIFAIPLGATEVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSAAFGGHSGANLIASDTTINGFSSFCVDTRQFTITLFYNVTNSYGYVSKSQDSNCPFTLQSVNDYLSFSKFCVSTSLLAGACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSNSTFNNTRELPGFFYHS

SEQ ID NO: 10 - DNA, 1392 bp, Artificial Sequence
  Synthetic construct
atggcgaacaagcacctgagccttagcctcttcctcgtgctcctgggcctctccgcctccctcgcctccggcgtttccttccaggacaggggcaggaagcgcgtgcccctctcgctctacgcgcccctcagggtcaccaacgacaagccgctcagcaaggtgctggccaataacgcggtccccacgaacaagggcaataaggaccagcagatcggctactggaacgagcagatccggtggaggatgaggaggggcgagaggatcgagcagccctccaactggcacttctactacctcggcacgggcccacacgcggacctgaggtaccgcaccaggacggagggcgtgttctgggtggccaaggagggcgctaagacggagcccaccaatctcggcgtgcgcaaggcctcggagaagccgatcatccccaacttctcccagcagctgccgagcgtggtcgagatcgtggagccaaacaccccgcccacctcgcgcgctaattcccgcagcaggtcccgcggcaacggcaacaataggagccgctccccgagcaacaacaggggcaataaccagtcccgcggcaacagccagaaccggggcaataaccagggcaggggcgccagccagaacaggggcggcaataacaacaataacaacaagtcccgcaatcagagcaagaacaggaaccagtcgaatgaccgcggcggcgtgacctcgcgcgacgacctcgtggcggccgtcaaggacgcgctcaagtccctgggcatcggcgagaacccggacaagctgaagcagcagcagaagccaaagcaggagaggtcggactccagcggcaagaacaccccgaagaagaataagtcccgcgccacgagcaaggagagggacctgaaggacatccccgagtggcgcaggatcccgaagggcgagaactcggtggcggcctgcttcggcccccgcggcggcttcaagaatttcggcgacgcggagttcgtggagaagggcgtggacgcttcgggctacgctcagatcgccagcctggctccaaacgtggcggccctcctgttcggtggcaacgtggcggtcagggagctggcggacagctacgagatcacctacaactacaagatgacggtgccgaagtccgaccccaatgtcgagctcctggtgagccaggtcgacgccttcaagaccggcaacgccaagccgcagcgcaagaaggagaagaagaacaagagggagacgacgcagcagctcaatgaggaggccatctacgacgacgtgggcgtcccatcggacgtgacgcacgccaacctggagtgggacaccgctgtcgacggcggggatactgctgtggagatcatcaacgagattttcgatactgggaattagtga

SEQ ID NO: 11 - protein, 462 aa, Artificial Sequence
  Synthetic construct
MANKHLSLSLFLVLLGLSASLASGVSFQDRGRKRVPLSLYAPLRVTNDKPLSKVLANNAVPTNKGNKDQQIGYWNEQIRWRMRRGERIEQPSNWHFYYLGTGPHADLRYRTRTEGVFWVAKEGAKTEPTNLGVRKASEKPIIPNFSQQLPSVVEIVEPNTPPTSRANSRSRSRGNGNNRSRSPSNNRGNNQSRGNSQNRGNNQGRGASQNRGGNNNNNNKSRNQSKNRNQSNDRGGVTSRDDLVAAVKDALKSLGIGENPDKLKQQQKPKQERSDSSGKNTPKKNKSRATSKERDLKDIPEWRRIPKGENSVAACFGPRGGFKNFGDAEFVEKGVDASGYAQIASLAPNVAALLFGGNVAVRELADSYEITYNYKMTVPKSDPNVELLVSQVDAFKTGNAKPQRKKEKKNKRETTQQLNEEAIYDDVGVPSDVTHANLEWDTAVDGGDTAVEIINEIFDTGN

SEQ ID NO: 12 - DNA, 558 bp, Artificial Sequence
  Synthetic construct
atggcgaacaagcacctgagccttagcctcttcctcgtgctcctgggcctctccgcctccctcgcctccggctcccacgagcagccgatcagcttcgtcacgctcccctccttcaacgaccacagcttcgtcaacatcaccgtgtccgcttccttcggtggccactcgggcgctaacctgatcgccagcgacaccacgatcaacggcttctccagcttctgcgtggacacgaggcagttcaccatctccctcttctacaacgtcacgaacagctacggctacgtgtccaagagccaggactccaactgcccgttcaccctccagagcgtcaacgactacctgtccttcagcaagttctgcgtgtccacgagcctgctggcttcggcctgcaccatcgacctgttcggctaccccgagttcggctccggcgtcaagttcacgagcctctacttccagttcaccaagggcgagctcatcacgggcacccccaagccactggagggcgtcacggacgtgttctacccctcctaccacagcaccccacagcgcccctgatag

SEQ ID NO: 13 - DNA, 36 bp, Artificial Sequence
  DC3 nucletotide sequence
ttctacccctcctaccacagcaccccacagcgcccc

SEQ ID NO: 14 - DNA, 309 bp, Escherichia coli
gccccgcagtccatcaccgagctctgctccgagtaccacaacacccagatctacaccatcaacgacaagatcctctcctacaccgagagcatggccggcaagcgcgagatggtgatcatcaccttcaagtccggcgccaccttccaggtggaggtgccgggctcccagcacatcgactcccagaagaaggccatcgagcgcatgaaggacaccctccgcatcacctacctcaccgagaccaagatcgacaagctctgcgtgtggaacaacaagaccccgaactccatcgccgccatcagcatggagaac

SEQ ID NO: 15 - protein, 103 aa, Escherichia coli
APQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 16 - DNA, 3002 bp, Zea mays
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacac

SEQ ID NO: 17 - DNA, 2520 bp, Zea mays
gcgctccctgacgctgtcttgggagagctgcaagatgagacactccatcccgcgcagccctgtcgtggcgtcctcctggatggacacctgcatcgctgtcgccctccaccaactcacctgaacgaagaatagaataaaaaatggagggagctgagggggcagtggttgcgctgtagggaggagagagaccgcgtcattataagactatctgcaaccgttacctctaaatttttccctctatatcattttttccccatattttcccccctattttttcatctcccgcaacggtttctcctaaatactccccctatatctcactaccactataaaatattattttttataccaactatcaattttttatctactaacaattactcgtggacccacagcacagtgtttaggagatgaacagtgacacgctatatctggggggagagagaaagaggccggcgcgtagggggcgccgtaggggcactgctgcggctgtagagtaccccctacacgccgcatgcaagggaagggggctgtcaggggggcaatgttgcgcatagcctaaagagcggatgaagcggcttgcaatttgcacgctggattcataaatagtgcatattactaaaaaaaagggtggggataggtatagagagtctattagagttgatctaagacccggtttatttcagattataatctgtccggattatataatccagcgcaaataatacagtaggtaaacaaacaactagattatgggttcagattatataatctaaaccccagattatgataatctcataatctcctcaagagtagcttattggagattattttggcaaaagacccactacccatggttatgtaaatagaaattataatatatatcatcttttttctcaccttaaataaacaaataagggtattgttgtctttatgaataatctacatttgtataatctaaactaccaaacaactacatctagattataatctggattatataatttaaattataatctagattatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccccttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacacc

SEQ ID NO: 18 - DNA, 6510 bp, Artificial Sequence
  Synthetic construct
ggcgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactaca

SEQ ID NO: 19 - DNA, 12 bp, Artificial Sequence
  Endoplasmic reticulum signal sequence
aaggacgagctc

SEQ ID NO: 20 - DNA, 28038 bp, Porcine epidemic diarrhea virus
acttaaaaagattttctatctacggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaactagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccmtgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactctcttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatggatacac

SEQ ID NO: 21 - protein, 1386 aa, Porcine epidemic diarrhea virus
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 22 - protein, 1386 aa, Porcine epidemic diarrhea virus
  misc_feature (728)..(728): Xaa can be any naturally occurring amino acid
  misc_feature (905)..(905): Xaa can be any naturally occurring amino acid
MRSLIYFWLSLPVLPTLSLPQDVYRCSARTNFRRFFSKFNVQAPAVVVLGGYLPSGETQGGAPRWYCAGRHETASGVHGIFLSHIRGGHGFEIGISQEPLDPSGYQLYLHKATNGNTNATARLRICQFPSNKTLGPTANDDVTTGRNCLFNKAIPAHMSEHSVVGITWDNDRVTVFADKIYHFYLKNEWSRVATKCYNSGGCAMQYVYEPIYYMLNVTSAGEDGISYQPCTANCIGYAANVFATESNGHIPEGFSFNNWFLLSNDSTLFHGKVVSNQPLLVNCLWAIPKIYGLGHFFSFNQTMDGVCNGATAYRAPEALRFNINDTSVILAEGSIVLHTALGTNLSFVCSNSSDPHKAIFSIPLGATQVPYYCFLKVDTYNSTVYKFLAVLPPTVREIVITKYGDVYVNGFGYLHLGLLDAVTINFTGHGTNDDVSGFWTIASTNFIDALVEVRATAIQRILYCDDPVCQLKCSQVSFDLDDGFYPISSRNLLSHEQPISFVTLPSFNDHSFVNITVSAAFGDSGGANLVASDTTINGFSSFCVDTRQFTIRLFYNVTSSYGYVSKSQDSNCPFTLQSVNDYLSFSKFCVSTSLLAGACTIDLFGYPAFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGGISSLSNSTFNNXRDFPGFFYHSNDGSNCTEPVLVYSNIGVCKSGSIGYVPLQDGQVKIAPTVIGNISIPTNFSMSIRTEYLQLYNTPVSVDCATYVCNGNSRCKQLLTQYTAACKTIESALELSARLESVEVNSMLTISEEALQLATISSFNGDGYNFTNVLGVSVYDSESGRVVHERSFIEDLLFNKXVTNGLGTVDEDYKRCSNGRSVADLVCAQYYSGIMVLPGVVDAEKLHMYSASLIGGMALGGFTAAVALPFSYAVQARLNYLALQTDVLQRNQQLLAESFNSAIGSITSAFESVNEAISQTSKGLNTVAHALTKVQEVVNSQGSALTQLTIQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIAGLCVNGDIALTLREPGLVLFTHELQTHTATEYFVSSRRMFEPRKPTVSDFVQIQSCVVTYVNLTSDQLPDVIPDYVDVNKTLDEILASLPNRTGPNLPLDVFNATYLNLTGEIADLEQRSESLRNTTEELRSLIYNINNTLVDLEWLNRVETYIKWPWWVWLIVFIVLIFVVSLLVFCCISTGCCGCCGCCGACFSGCCRGPRLQPYEAFEKVHVQ

SEQ ID NO: 23 - protein, 1382 aa, Porcine epidemic diarrhea virus
MTPLIYFWLFLPVLLTLSLPQDVTRCQSTINFRRFFSKFNVQAPAVVVLGGYLPSMNSSSWYCGTGIETDSGVHGIFLSYIDSGQGFEIGISQEPFDPSGYQLYLHKATNGNTSAIARLRICQFPDNKTLGPTVNDVTTGRNCLFNKAIPALQDGKNIVVGITWDNDRVTVFADKIYHFYIKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLRAIPKIYGLGQFFSFNQTMDGVCNGAAAQRAPEALRFNINDTFVILAEGSIVLHTALGTNLSFVCSNSSDPHKAIFTIPLGVTEVPYYCFLKVDTYKSTVYKFLAVLPPTVKEIVITKYGDVYVNGFGYLHLGLLDAVTINFTGHGTDDDVSGFWTVASTNFVDALIEVQGTAIQRILYCDDPVSQLKCSQVSFDLDDGFYPISSRNLLSHEQPISFVTLPSFNDHSFVNITVSAAFGGHSGANLIASDTTTNGFSSFCVDTRQFTITLFYNVTNSYGYVSKSQDSNCPFTLQSVNDYLSFSKFCVSTSLLAGACTIDLFGYPEFGSGVKFTSLYFQFTKGELITGTPKPLQGVTDVSFMTLDVCTKYTIYGFKGEGIITLTNSSFLAGVYYTSDSGQLLAFKNVTSGAVYSVTPCSFSEQAAYVDDDIVGVISSLSNSTFNNTRELPGFFYHSNDGSNCTEPVLVYSNIGVCKSGSIGYVPLQDGQVKIAPMVTGNISIPTNFSMSIRTEYLQLYNTPVSVDCVTYVCNGNSRCKQLLTQYTAACKTIESALQLSARLESVEVNSMLTISEEALQLATISSFNGDGYNFTNVLGVSVYDPASGRVVQKGSFIEDLLFNKVVTNGLGTVDEDYKRCSNGRSVADLVCAQYYSGVMVLPGVVDAEKLHMYSASLLGGMALGGLTTAAALPFSNAVQARLNYLALQTDVLQRNQQLLAESFNSAIGNITSAFESVKEAISQTSNGLNTVAHALTKVQEVVKSQGSALTQLTIQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIDGLCVNGDIALTLREPGLVLFTHELQTYTATEYFVSSRRMFEPRKPTVSDFVQIESCVVTYVNLTSDQLPDVIPDYIDVNKTLDEILASLPNRIGPSLPLDVFNATYLNLTGEIADLEQRSESLRNTTEELRSLIYNINNTLVDLEWLNRVETYIKWPWWVWLIIFIVLIFVVSLLVFCCISTGCCGCCGCCGACFSGCCRGPRLQPYEAFEKVHVQ

SEQ ID NO: 24 - protein, 4 aa, Artificial Sequence
  Endoplasmic reticulum signal sequence
HDEL

SEQ ID NO: 25 - DNA, 2217 bp, Artificial Sequence
  Synthetic construct
atggcgaacaagcacctgagccttagcctcttcctcgtgctcctgggcctctccgcctccctcgcctccggcgtcactcgctgccagtcgaccatcaacttccgcaggttcttctccaagttcaacgtgcaggcccccgcggtggtggtgctcggcggctacctgccctccatgaattctagctcctggtactgcggcacgggcatcgagacggactcgggcgtgcacggcatcttcctcagctacatcgactccggccagggcttcgagatcggcatctcgcaggagcccttcgacccatcgggctaccagctctacctgcacaaggccaccaacggcaacacgaacgcgatcgcccgcctcaggatctgccagttcccggacaacaagaccctgggcccaaccgtcaacgacgtgaccaccggccgcaactgcctgttcaacaaggcgatcccggcctacctccaggacggcaagaacatcgtcgtgggcatcacgtgggacaacgacagggtcaccgtgttcgcggacaagatctaccacttctacctcaagaacgactggtcccgcgtcgcgaccaggtgctacaacaagcgcagctgcgccatgcagtatgtgtacaccccgacgtactacatgctcaacgtgacctcggcgggcgaggacggcatctactacgagccgtgcacggccaactgctcgggctacgcggccaacgtgttcgctaccgactccaacggccacatcccggagggcttcagcttcaacaactggttcctcctgagcaacgactccaccctcctgcacggcaaggtcgtgtccaaccagccgctcctggtcaactgcctcctggcgatccccaagatctacggcctgggccagttcttcagcttcaaccagacgatggacggcgtgtgcaacggcgcggccgctcagagggcccccgaggccctcaggttcaacatcaacgacacgagcgtcatcctggccgagggctccatcgtgctccacacggctctgggcacgaacctcagcttcgtgtgctccaacagctccgacccccacctggccatcttcgctatccccctgggcgccacggaggtcccctactactgcttcctgaaggtggacacctacaacagcacggtctacaagttcctggcggtgctgccgcccacggtcagggagatcgtgatcaccaagtacggcgacgtctacgtgaacggcttcggctacctccacctgggcctcctggacgcggtcaccatcaacttcaccggccacggcacggacgacgacgtgagcggcttctggacgatcgcgagcaccaacttcgtggacgctctcatcgaggtgcagggcaccgcgatccagaggatcctgtactgcgacgacccggtcagccagctcaagtgctcccaggtggccttcgacctggacgacggcttctaccccatcagctcccgcaacctcctgagccacgagcagccgatctccttcgtcaccctgcccagcttcaacgaccactccttcgtcaacatcacggtgagcgccgctttcggtggccactcgggcgccaacctcatcgcttccgacaccacgatcaacggcttcagctccttctgcgtggacacgcgccagttcaccatcacgctgttctacaacgtcaccaactcctacggctacgtgagcaagtcccaggactccaactgcccgttcaccctgcagagcgtcaacgactacctcagcttctccaagttctgcgtgagcacgtccctcctggccggcgcttgcaccatcgacctcttcggctaccccgagttcggcagcggcgtcaagttcacgtccctgtacttccagttcaccaagggcgagctgatcacgggcacccccaagccactggagggcgtcacggacgtgagcttcatgaccctcgacgtgtgcaccaagtacacgatctacggcttcaagggcgagggcatcatcaccctgacgaactcaagctttctcgccggcgtctactacaccagcgactccggccagctcctggcgttcaagaacgtgacctcgggcgcggtctactcggtgacgccctgcagcttctccgagcaggccgcctacgtggacgacgacatcgtcggcgtgatcagctccctgagcaactcaacattcaacaacactcgggagctgcctggcttcttctaccattcctgatag

(19) United States
(12) Patent Application Publication
(10) Pub. No.: US 2022/0387581 A1
(43) Pub. Date: Dec. 8, 2022

(54) ORAL ADMINISTRATION OF CORONAVIRUS SPIKE PROTEIN FOR ALTERING CYTOKINE LEVELS AND PROVIDING PASSIVE IMMUNITY TO NEWBORN PIGS

(71) Applicant: MAZEN ANIMAL HEALTH INC., Ames, IA (US)

(72) Inventors: John Howard, San Luis Obispo, CA (US); Gina Fake, San Luis Obispo, CA (US)

(21) Appl. No.: 17/805,396

(22) Filed: Jun. 3, 2022

Related U.S. Application Data
(60) Provisional application No. 63/202,264, filed on Jun. 3, 2021.

(51) Int. Cl. A61K 39/215; A61K 9/00; A61P 31/14; A61P 37/06; A23K 10/30; A23K 20/147; A23K 50/30

(52) U.S. Cl. CPC A61K 39/215; A61K 9/0053; A61P 31/14; A61P 37/06; A23K 10/30; A23K 20/147; A23K 50/30; A61K 2039/545

(57) ABSTRACT

Plants and plant produced compositions which include Coronavirus S proteins are disclosed. These may be used as vaccines, boosters or immune modulators. The compositions have been shown to reduce the inflammatory cytokine response by altering cytokine levels when administered to an animal. The compositions may be used as an immune modulator to reduce/ameliorate or prevent the cytokine storm often associated with Coronavirus or other virus infection. The compositions may also be used to produce additive protection when administered with any vaccine composition to increase vaccine effectiveness. The compositions when used as vaccines have been shown to protect newborn animals through passive immunity.

### CROSS-REFERENCE TO RELATED APPLICATIONS

This application claims priority under 35 U.S.C. § 119 to provisional application Ser. No. 63/202,264, filed Jun. 3, 2021, herein incorporated by reference in its entirety.

### INCORPORATION OF SEQUENCE LISTING

A sequence listing containing the file named “HOWARD_P13625US01_SEQ_LISTING_ST25.txt” which is 130,456 bytes (measured in MS-Windows®), comprises 111 biological sequences, and was created on Jun. 2, 2022, is electronically filed herewith via the USPTO's Patent Center system, and is incorporated herein by reference in its entirety.

### BACKGROUND

Coronaviruses have become a major problem for both human and animal welfare and are a continuing threat in the near future. Porcine Epidemic Diarrhea Virus (PEDV) is a positive strand enveloped RNA virus of family Coronaviridae with a genome of 28 kb PEDV. The virus infects swine resulting in major losses to the industry in the US and worldwide (Gerdts and Zakhartchouk, 2017). Newborn piglets are especially susceptible with a high mortality rate. The disease was first identified in Europe in the early 1970s, in Asia in 2010, and in the United States in 2013. It continues to be a major problem in the swine industry.

PEDV causes severe diarrhea and mortality in piglets. Vaccines have the potential to provide a robust immunity and break the transmission cycle and while there have been promising results, none have provided complete protection from the virus. The spike protein (S) is the target for most vaccine strategies for coronavirus as it contains the majority of epitopes for neutralizing antibodies. In the case of PEDV, nursing piglets are in most need of protection, but they cannot mount their own immune response in time and must rely on passive immunity.

Vaccines based on the market from sources such as Harris vaccines and Zoetis, but are only marginally effective and are largely based on classical strains such as CV777 (Gerdts and Zakhartchouk, 2017). There is a clear need for a low-cost and more effective vaccine for PEDV.

### SUMMARY

Applicants have discovered benefits of coronavirus vaccines, booster compositions or immune modulating compositions comprising coronavirus spike protein (S protein) produced from plants. Plant produced or plant based vaccines, booster compositions or immune modulating from Porcine Epidemic Diarrhea Virus (PEDV) are demonstrated herein to provide passive immunity to newborn animals from mothers vaccinated and also to alter cytokine levels making the vaccine more effective which may be used as a booster for any vaccine. Briefly for production of the vaccine a construct is introduced into a plant comprising a promoter preferentially directing expression to seed of the plant, a nucleic acid molecule encoding a Spike polypeptide of PEDV and a nucleic acid molecule targeting expression to the endoplasmic reticulum. Embodiments provide the construct with a sequence of S protein including the COE polypeptide, a sequence encoding the LTB heat labile polypeptide or a combination thereof. Expression levels of at least 10 mg/kg of seed of the plant are obtained. When the plant or plant product is orally administered to the animal, a protective response is observed, including a serum antibody response. Further benefits include altered cytokine levels to reduce nonspecific immune reactions and passive immunity.

Applicants administered the S antigen to naïve sows and gilts which were then boosted prior to farrowing. The newborn pigs were then challenged with PEDV and evaluated for disease symptoms. Nursing piglets from dams having had the oral vaccine candidate showed significantly higher survival rates than controls. In addition, dams were tested for correlates of protection. Milk and sera neutralizing antibodies (NABs) showed a significant correlation. The levels of thirteen different cytokines were also measured in dams and found in most cases to have reduced levels when administered with the S antigen compared to control dams. For INF, the cytokine level was increased which is desirable for reducing the non-specific immune response. This demonstrates that the S antigen from coronavirus may act not only as an immunogen to elicit (NABs) but may also act as an immune modulator to decrease cytokine levels and reducing the inflammatory response prior to viral exposure leading to reduction in disease severity Thus, the S protein and compositions containing the same may be used as an immune modulator to reduce/ameliorate or prevent the cytokine storm often associated with Coronavirus or other virus infection. The compositions may also be used to produce additive protection when administered with any vaccine composition to increase vaccine effectiveness. The effects are seen upon oral or injection administration and in some embodiments may be used as an oral accompaniment to an injected vaccine or in a completely oral vaccine protocol.

In a further embodiment the cytokine levels which are increased post exposure to Coronavirus may be used as a marker to indicate the presence of immunity or as a diagnostic for exposure to the same.

### DESCRIPTION OF THE FIGURES

FIG. 1 is a graphic showing constructs created for introduction into plants. Promoters used were pr25, pr39 and pr44. BAASS refers to the barley alpha amylase sequence, S1 refers to the US strain spike proteins where S1ext(US) refers to an extended sequence and S1(DR13) refers to a South Korean strain spike protein; Vac refers to a vacuoletargeting sequence, KDEL refers to an endoplasmic reticulum retaining sequence; COE refers to the COE sequence; Pinll is the Pinll terminator and LTB refers to the heat labile enterotoxin subunit, all of which are described in further detail herein.

FIG. 2 is a graph showing piglet health after viral challagne over time.

FIG. 3 is a graph showing mean survival rates of piglets per group. The results show the high mortality rate of the controls while the orally administered vaccine candidate provided the greatest protection.

FIG. 4 is a graph showing NABs in Sera for injected, control and oral delivery. Results are given in the highest titer that provided a positive result with a dilution of 20 being the limit of detection for a positive sample.

FIG. 5 is a graph depicting cytokine levels in sow's milk. The levels for the 13 different cytokines shown were determined, means calculated and then the injected and orally administered group were compared to the control group of sows. Overall, there was a marked decrease in cytokine levels for sows that had been administered the S protein either orally or parenterally.

FIG. 6 is a graph showing cytokines in sow's sera. The levels for the 13 different cytokines shown were determined, means calculated and then orally administered group was compared to the control group of sows. Overall, there was a marked decrease in cytokine levels for sows that had been administered the S protein.

FIG. 7 is a graph showing the mean survival rates of the six different treatment groups.

FIG. 8 is a graph showing the levels of cytokines in milk for all six treatment groups. The data demonstrates that CSF, IFN and TNF all are decreased in milk from controls and from injected administration when orally administered the S protein composition.

### DESCRIPTION

Porcine Epidemic Diarrhea Virus (PEDV) is a member of the subfamily Coronavirinae of genus Alphacoronavirus (Bridgen et al. 1993 Sequence determination of the nucleocapsid protein gene of the porcine epidemic diarrhoea virus confirms that this virus is a coronavirus related to human coronavirus 229E and porcine transmissible gastroenteritis virus. J. Gen. Virol. 74 (Pt 9):1795-1804) and was first identified in England in 1971 and later in other countries, such as Belgium, China, Hungary, Italy, Japan, Korea, and Thailand (Oldham J. 1972 Letter to the editor. Pig Farming 1972 (October suppl):72-73; Pensaert and De Bouck P. 1978 A new coronavirus-like particle associated with diarrhoea in swine. Arch. Virol. 58:243-247; Molecular characterization and phylogenetic analysis of membrane protein genes of porcine epidemic diarrhea virus isolates in China. Virus Genes 36:355-364; Nagy et al. 1996. Enterotoxigenic Escherichia coli, rotavirus, porcine epidemic diarrhea virus, adenovirus and calici-like virus in porcine postweaning diarrhoea in Hungary. Acta Vet. Hung. 44:9-19; Martelli et al. 2008. Epidemic of diarrhoea caused by porcine epidemic diarrhoea virus in Italy. Vet. Rec. 162:307-310; Takahashi et al. 1983. An outbreak of swine diarrhea of a new-type associated with coronavirus-like particles in Japan. Nippon Juigaku Zasshi 45:829-832; Chae et al. 2000. Prevalence of porcine epidemic diarrhoea virus and transmissible gastroenteritis virus infection in Korean pigs. Vet. Rec. 147:606-608; Puranaveja et al. 2009. Chinese-like strain of porcine epidemic diarrhea virus, Thailand. Emerg. Infect. Dis. 15:1112-1115). Other members of this family include Porcine Respiratory Coronavirus (PRCV), Hemagglutinating Encephalomyelitis Coronavirus (PHE), and Transmissible Gastroenteritis Virus (TGEV). Although PEDV and TGEV viruses are related and the clinical signs are very similar, there is no immune cross-protection.

PEDV is an enveloped virus possessing approximately a 28 kb, positive-sense, single stranded RNA genome, with a 5′ cap and a 3′ polyadenylated tail. (Pensaert and De Bouck P. 1978). The genome comprises a 5′ untranslated region (UTR), a 3′ UTR, and at least seven open reading frames (ORFs) that encode four structural proteins (spike (S), envelope (E), membrane (M), and nucleocapsid (N)) and three non-structural proteins (replicases 1a and 1b and ORF3); these are arranged on the genome in the order 5′-replicase (1a/1b)-S-ORF3-E-M-N-3′ (Oldham J. 1972; and Bridgen et al. 1993). The first three emergent North American PEDV genomic sequences characterized, Minnesota MN (GenBank: KF468752.1), Iowa IA1 (GenBank: KF468753.1), and Iowa IA2 (GenBank: KF468754.1), have the same size of 28,038 nucleotides (nt), excluding the polyadenosine tail and share the genome organization with the prototype PEDV CV777 strain (GenBank: AF353511.1). These three North American PEDV sequences shared 99.8 to 99.9% nucleotide identities. In particular, strains MN and IA2 had only 11 nucleotide differences across the entire genome.

The PEDV Spike (S) protein is a type I glycoprotein composed of about 1,383 amino acids (aa) (1386 with the Korea strain, see e.g., GenBank Ref NO. AAM19716.1 (SEQ ID NO: 22), identifying the S1 region as residues 234-736; 1382 amino acids in a China strain, with S1 identified as 230-732, see GenBank Ref No. AFL02627.1 (SEQ ID NO: 23)). It contains a putative signal peptide (residues 1-24). The S protein can be divided into two regions. One is the N-terminal region of S1 (1-733 or 735 aa). Referring to the Spike protein used in the example below, it has 94% identity to Korean strain example of AAM19716.1 and 93% identity with the China strain example of AFL02627.1. The other region is the C-terminal region S2 which is identified as including residues 736-741 to the end of the Spike protein based on its homology with S protein of other coronaviruses (Chang et al. 2002 Identification of the epitope region capable of inducing neutralizing antibodies against the porcine epidemic diarrhea virus. Mol. Cells 14, 295-299. Cleavage of the spike protein into S1 and S2 can occur in the presence of trypsin. (See e.g., Wicht et al. (2014) Proteolytic activation of the porcine epidemic diarrhea coronavirus spike fusion protein by trypsin in cell culture. J. Virol. 88:2952-7961). The GPRLQPY motif located at the carboxy-terminal of the spike protein induces antibodies that neutralize Porcine epidemic diarrhea virus. Godet et al. 1994Virus Res. 132, 192-196. Major receptor-binding and neutralization determinants are located within the same domain of the transmissible gastroenteritis virus (coronavirus) spike protein. J. Virol. 68, 8008-8016; Jackwood et al. 2001. Spike glycoprotein cleavage recognition site analysis of infectious bronchitis virus. Avian Dis. 45, 366-372; Sturman et al. 1984 Proteolytic cleavage of peplomeric glycoprotein E2 of MHV yields two 90K subunits and activates cell fusion. Adv. Exp. Med. Biol. 173, 25-35. 33; Sun et al. 2008. Identification of two novel B cell epitopes on porcine epidemic diarrhea virus spike protein. Vet. Microbiol. 131, 73-81. 34.). The S protein in coronaviruses is a surface antigen, where it plays a role in regulating interactions with host cell receptor glycoproteins to mediate viral entry and stimulating induction of neutralizing antibodies in the natural host. A phylogenetic and genetic comparison analysis of the S gene and its regions showed minor variations among strains, including the US, China, Korea, showed a percent identity ranging from 89.4% to 100% identity. This included percent identity of comparison of strains DR13, BM1, J3142, BM3, CV777, AH2012, BJ-2012-2, Colorado30, Indiana34 and Texas 31. See Chung et al (2017) Genetic characterization of S1 domain of porcine epidemic diarrhea viruses spike proteins isolated in Korea, J. Immune Disord. Vol. 1 No. 1. Sequence comparisons of the polypeptide of the S protein showed Korean isolates had 93.6% to 99.6% identity with each other and 92.2%-93.7% identity with other strains. Lee et al. (2010) Hetergeneity in spike protein genes of porcine epidemic diarrhea viruses isolate din Kore, Virus Res. 149(2):175-82. Thus, the S glycoprotein is a primary target for the development of effective vaccines against PEDV.

The PEDV M protein is the most abundant envelope component playing an important role in the viral assembly process and also induces antibodies that neutralize the virus. Likewise, the PEDV N protein, which binds to virion RNA providing a structural basis for the nucleocapsid, may also be important for induction of cell-mediated immunity (Saif, L. 1993 Coronavirus immunogens. Vet. Microbiol. 285-297.).

The only accessory gene in PEDV is ORF3. While accessory genes are generally maintained in field strains, alteration of ORF3 is thought to influence virulence; cell culture adaptation has been used to alter the ORF3 gene in order to reduce virulence (Song et al. 2003 Differentiation of a Vero cell adapted porcine epidemic diarrhea virus from Korean field strains by restriction fragment length polymorphism analysis of ORF 3. Vaccine 21, 1833-1842). In fact, through investigation of the ORF3 gene, researchers have charted the emergence of new genogroups of PEDV in immunized swine herds in China since 2006. Phylogenic studies of these strains and the geographical reemergence of PEDV in China have demonstrated that those field strains causing devastating enteric disease differ genetically in ORF3 from the European strains and vaccine strains (Park et al. 2011) Molecular characterization and phylogenetic analysis of porcine epidemic diarrhea virus (PEDV) field isolates in Korea. Arch. Virol. 156, 577-585.

Different strains of PEDV exist with varying levels of virulence. The clinical signs of PEDV infection are similar to transmissible gastroenteritis virus (TGEV) infection (Pijpers et al. 1993). In pigs three weeks of age and younger, clinical signs (including acute watery, diarrhea, vomiting, and dehydration) can be seen as soon as 24 hours after PEDV infection leading to 100% mortality. PEDV-infected feeder and grower pigs, as well as sows and boars, can develop diarrhea and vomiting. The animals can also show signs of anorexia and can be lethargic. Older pigs show reduced feed efficiency, additional days to market, and the susceptibility of infected animals to secondary infections is likely. For sows, reduced body condition may negatively impact reproductive performance.

The gross and histological changes in the gut of animals infected with PEDV are similar in the United States as those observed in China; essentially the virus destroys the villi of a pig's intestine so that there is a failure to absorb nutrients. Animals succumbing to the disease in the Minnesota and Iowa outbreaks had gross pathological lesions confined to the small intestine and that the small intestine was characterized by thin translucent walls distended with yellow fluid. Histological evaluations revealed regions of small intestines with villus blunting and fusion and minimal lymphoblastic infiltration of the villi of the lamia propria.

Huang et al. 2013 characterized three different strains of PEDV from outgoing outbreaks in the United States—one from Minnesota and two from Iowa, designated MN (GenBank accession No: KF468752) and IA1 (GenBank accession No: KF468753) and IA2 (GenBank accession No: KF48754), respectively. (Huang et al. 2013 Origin, evolution, and genotyping of emergent porcine epidemic diarrhea virus strains in the United States. mBio 4(5):e00737-13.) Huang's phylogenic survey grouped PEDV strains as falling into two distinct genogroups, designated genogroup 1 (G1) and genogroup 2 (G2). Genogroup 1 includes at least three clusters 1a, 1b, and R. Subgroup 1a includes the early European, Chinese, and Korean isolates, e.g., prototype CV777 strain (Belgium, 1978, GenBank: AF353511.1) and strains LZC (Gansu, China, 2006; GenBank: EF185992) and SM98 (Korea, 1998; GenBank: GU937797.1). Subgroup 1b contains five strains—one from South Korea (the DR13 attenuated vaccine strain, GenBank: JQ023162.1) and the others from China linked by the common “genetic signature” 8-aa deletion in nsp3 and the large ORF3 deletion at the C terminus. Group “R” is associated with recombinants of the other genogroups. Certain strains belong to genogroup G2a. The Chinese strain AH2012 (GenBank accession no: KC210145) and the North American strains share several unique nucleotides changes and are clustered together in genogroup 2a. Nucleotide identity to AH2012 for strains MN and IA2 was 99.6% and for strain IA1 was 99.5%. A closely related North American isolate US/Colorado/2013 (GenBank Accession No: KF272920.1) has also been reported by Marthaler et al, 2013 Complete genome sequence of porcine epidemic diarrhea virus strain USA/Colorado/2013 from the United States. Genome Announc. 1(4):e00555-13.10.1128/genomeA.00555-13. Like the North American isolates above, the complete PEDV genome of CO/13 has a nucleotide identity of 96.5 to 99.5% with other complete PEDV genomes available in GenBank, with the highest nucleotide identity (99.5%) with Chinese strain AH2012 (GenBank Accession No. KC210145). It is a member of the 2a genogroup.

Attempts to create PEDV vaccines include production of attenuated viral vaccines, such as that described at U.S. Pat. No. 9,950,061, incorporated by reference in its entirety. The attenuated vaccine included a Spike antigen, with that modified Spike protein shown as sequence identifier 9, encoded by the nucleic acid of sequence identifier 8 with variations effective for protection shown having at least 80% homology and included sequence identifiers 3, 7, 9 and 14, all of which are incorporated by reference herein in their entirety.

Here is provided a plant-produced Spike (S) polypeptide and it use as an immune modulator and compositions comprising the same. In an embodiment the S polypeptide is introduced into a plant in a construct comprising a seed-preferred promoter which may further prefer expression to the embryo of the seed, operably linked to the nucleic acid molecule encoding the S polypeptide. In further embodiments the construct comprises nucleic acid molecules that retain expression of the S polypeptide in the endoplasmic reticulum of the cell of the plant. Still further embodiments provide for two plant transcription units (PTUs) with each PTU comprising an embryo preferred promoter and nucleic acid molecules retaining expression in the endoplasmic reticulum. Additional embodiments provide the PTUs comprises the same seed preferred promoter and nucleic acid molecules retaining expression in the endoplasmic reticulum.

The coronavirus viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome. Coronavirus Spike (S) protein is class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain. In some embodiments, the coronavirus is a Severe Acute Respiratory Syndrome (SARS)-coronavirus (SARS-CoV-1), a SARS-coronavirus 2 (SARS-CoV-2), a SARS-like coronavirus, a Middle East Respiratory Syndrome (MERS)-coronavirus (MERS-CoV), a MERS-like coronavirus, NL63-CoV, 229E-CoV, 0C43-CoV, HKU1-CoV, WIV1-CoV, MHV, HKU9-CoV, PEDV-CoV, or SDCV. In any of the preceding embodiments, the S protein can comprise a coronavirus spike (S) protein or a fragment or epitope thereof, wherein the epitope is optionally a linear epitope or a conformational epitope, and wherein the protein comprises three recombinant polypeptides. In any of the preceding embodiments, the surface antigen can comprise a signal peptide, an S1 subunit peptide, an S2 subunit peptide, or any combination thereof.

The S protein is expressed poorly in recombinant systems, therefore, it is difficult to develop a commercial subunit vaccine. Here in an embodiment, maize grain is used as a basis for the production of the subunit vaccine. High expression levels of at least 10 mg/kg of whole seed are obtained. An embodiment provides for a range of about 10-100 mg/kg. Further embodiments provide for expression at llmg/kg, 12 mg/kg, 13, 14, 15, 16, 17, 18, 19, 20, 25, 30, 35, 40 mg/kg of whole seed or more or amounts in-between. See Published application number US 2020-0080101A published Mar. 12, 2020 entitled EXPRESSION OF PEDV SEQUENCES IN PLANTS AND PLANT PRODUCED VACCINE FOR SAME, the disclosure of which is hereby incorporated by reference.

Further, oral administration of the plant, plant part or a product produced from the plant part, such as a seed, grain, flour or other edible composition comprising the plant, plant part or product produced therefrom comprising the Spike protein results in surprising serum response from animals and can also produce a mucosal response as well. The serum response in an embodiment is within the range of two-100 fold more than the control. In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals not receiving vaccination, or amounts in-between.

As used herein, the terms nucleic acid or polynucleotide refer to deoxyribonucleotides or ribonucleotides and polymers thereof in either single- or double-stranded form. The sequence used to make the vaccine may be obtained from any source, such as a biological source in isolating from a biological sample or can refer to a sequence synthetically produced based upon the sequence obtained from the sample. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

Nucleic acids employed here include those that encode an entire polypeptide as well as those that encode a subsequence of the polypeptide or produce a fragment that provides a protective response. For example, nucleic acids that encode a polypeptide which is not full-length but nonetheless has protective activity against PEDV. The invention includes not only nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%,94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The nucleotide sequence may be modified as described previously, so long as any polypeptide encoded produced is capable of inducing the generation of a protective response.

The nucleic acids can be obtained using methods that are known to those of skill in the art. Suitable nucleic acids (e.g., cDNA, genomic, or subsequences) can be cloned, or amplified by in vitro methods such as the polymerase chain reaction (PCR) using suitable primers, the ligase chain reaction (LCR), the transcription-based amplification system (TAS), or the self-sustained sequence replication system (SSR). A wide variety of cloning and in vitro amplification methodologies are well-known to persons of skill Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (2001) Molecular Cloning—A Laboratory Manual (Third ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C& EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomeli et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Nucleic acids or subsequences of these nucleic acids, can be prepared by any suitable method as described above, including, for example, cloning and restriction of appropriate sequences.

“Codon optimization” can be used to optimize sequences for expression in an animal and is defined as modifying a nucleic acid sequence for enhanced expression in the cells of the animal of interest, e.g. swine, by replacing at least one, more than one, or a significant number, of codons of the native sequence with codons that are more frequently or most frequently used in the genes of that animal. Various species exhibit particular bias for certain codons of a particular amino acid.

As used herein, a “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present invention, means a polypeptide which retains essentially the same biological function or activity as said polypeptide, that is, act as an antigen and/or provide treatment for and/or protection against disease. Such fragments, derivatives and homologues can be chosen based on the ability to retain one or more of the biological activities of the polypeptide, that is, act as an antigen and/or provide treatment for and/or protection against the pathogen. The polypeptide vaccines of the present invention may be recombinant polypeptides, natural polypeptides or synthetic polypeptides, preferably recombinant polypeptides. One skilled in the art appreciates that it is possible that the protective polypeptide may be expressed by the gene in the host cells and the plant composition administered to the animal or extracted from the plant prior to administration.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques. Many of these techniques are described in detail below.

The methods include amino acids that include an amino acid sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%,94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The sequence may be modified as described previously, so long the polypeptide is capable of inducing the generation of a protective response.

The variant proteins used in the present methods can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present methods further provide functional fragments of the nucleic acid molecules and polypeptides including variant proteins of the polyeptide, in addition to proteins and peptides that comprise and consist of such fragments, provided that such fragments act as an antigen and/or provide treatment for and/or protection against PEDV.

As used herein, the term “subunit” refers to a portion of the microorganism which provides protection and may itself be antigenic, i.e., capable of inducing an immune response in an animal. The term should be construed to include subunits which are obtained by both recombinant and biochemical methods.

A “construct” is a package of genetic material inserted into the genome of a cell via various techniques. A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo or in vivo. Viral vectors include alphavirus, retrovirus, adeno-associated virus, pox, baculovirus, vaccinia, herpes simplex, Epstein-Barr, rabies virus, vesicular stomatitis virus, and adenovirus vectors. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A nucleic acid molecule is introduced into a cell when it is inserted in the cell. A cell has been “transfected” by exogenous or heterologous DNA or RNA when such DNA or RNA has been introduced inside the cell.

A cell has been “transformed” by exogenous or heterologous DNA or RNA when the transfected DNA or RNA effects a phenotypic change. The transforming DNA can be integrated (covalently linked) into chromosomal DNA making up the genome of the cell.

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus, the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to a seed or an embryo preferred promoter is meant that it expresses an operably linked sequence to a higher degree in seed or embryo tissue that in other plant tissue. It may express during seed or embryo development, along with expression at other stages, may express strongly during seed or embryo development and to a much lesser degree at other times.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991)Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. There are a wide variety of endoplasmic reticulum retention signal sequences available to one skilled in the art and the KDEL sequence is one example. Another example is HDEL (His-Asp-Glu-Leu (SEQ ID NO: 24)). See, for example, Kumar et al. which discuses methods of producing a variety of endoplasmic reticulum proteins. Kumar et al. (2017) “prediction of endoplasmic reticulum resident proteins using fragmented amino acid composition and support vector machine” Peer J. doi: 10.7717/peerj.3561.

Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

The termination region can be native with the promoter nucleotide sequence can be native with the DNA sequence of interest or can be derived from another source. Convenient termination regions are available from the Ti-plasmid of A. tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp 7831-7846 (nos). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

The term plant refers to the entire plant or plant material or plant part or plant tissue or plant cell including a collection of plant cells. It is used broadly herein to include any plant at any stage of development, or to part of a plant, including a plant cutting, a plant cell culture, a plant organ, a plant seed, and a plantlet. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

Any plant species may be used, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardiurn occidentale), macadamia (Macadamia integrifolia), almond (Prunus amygdalus), sugar beets (Beta vulgaris), oats(Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, algae or Lemnoideae (aka duckweed), pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). An embodiment provides the plant is maize.

The method of transformation/transfection is not critical; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993). Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hi II maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase and are harvested at a density of no more than OD₆₀₀=0.5, preferably between 0.2 and 0.5. The bacteria are then centrifuged to remove the media and resuspended in the co-culture medium. Since Hi II is used, medium preferred for Hi II is used. This medium is described in considerable detail by Armstrong and Green (1985). The resuspension medium is the same as that described above. All further Hi II media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity and becomes “reprogrammed” to have a new identity. Thus, the scutellum cells become embryogenic callus.

A transgenic plant may be produced that contains an introduced nucleic acid molecule encoding the polypeptide.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn., 4^{th} Edit.). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Selection and propagation techniques described above can yield a plurality of transgenic plants that are harvested in a conventional manner. The plant or any parts expressing the recombinant polypeptide can be used in a commercial process, or the polypeptide extracted. When using the plant or part itself, it can, for example, be made into flour and then applied in the commercial process. Polypeptide extraction from biomass can be accomplished by known methods. Downstream processing for any production system refers to all unit operations after product synthesis, in this case protein production in transgenic seed (Kusnadi, A. R., Nikolov, Z. L., Howard, J. A., 1997. Biotechnology and Bioengineering. 56:473-484). For example, seed can be processed either as whole seed ground into flour or, fractionated and the germ separated from the hulls and endosperm. If germ is used, it is usually defatted using an extraction process and the remaining crushed germ ground into a meal or flour. In some cases, the germ is used directly in the process, or the protein can be extracted (See, e.g., WO 98/39461). Extraction is generally made into aqueous buffers at specific pH to enhance recombinant protein extraction and minimize native seed protein extraction. Subsequent protein concentration or purification can follow.

The therapeutics of the invention can be tested in vitro for the desired therapeutic or prophylactic activity, prior to in vivo use in animals. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutics may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

In addition, the therapeutics can be assessed by measuring the level of the molecule against which the antibody is directed in the animal model or human subject at suitable time intervals before, during, or after therapy. Any change or absence of change in the amount of the molecule can be identified and correlated with the effect of the treatment on the subject. The level of the molecule can be determined by any method known in the art.

The following is provided by way of illustration within intending to be limiting of the scope of the invention. All references cited herein are incorporated herein by reference.

Embodiments of the invention include the following:

1. A method of providing passive immunity protection to an animal from the effects of Coronavirus introduction, comprising:
administering to said animal prior to farrowing a composition of a plant or plant product comprising the Spike (S1) protein of Coronavirus.
2. The method of claim 1, wherein said Coronavirus introduction is via oral administration
3. The method of claim 1 wherein said Coronavirus is PEDV.
4. The method of claim 1 where the S protein is fused to another protein.
5. The method of claim 3 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that inflammatory cytokine levels are altered to reduce inflammation prior to infection.
6. The method of claim 1 wherein said animal is a dam.
7. The method of claim 1 wherein said administration includes a booster prior to farrowing.
8. The method of claim 6 wherein said dam passes protection to her piglets.
9. The method of claim 1 wherein is said administration is 3 doses.
10. The method of claim 1 wherein said plant or plant product is seed which expresses said S Protein.
11. The method of claim 8 wherein said seed is dosed with animal feed.
12. The method of claim 1 wherein said composition decreases cytokine inflammatory response by altering cytokine levels in said animal.
13. The method of claim 1 wherein said cytokine level that is altered includes one or more of GM-CSF, IFN gamma, IL-lalpha, IL-1beta, IL-1ra, IL-2, IL-4, IL-6, IL-8, IL-10, IL-12, IL18, or TNF alpha.
14. The method of claim 13 wherein said cytokine level is one or more of GN-CSF, IFN gamma, and/or TNF alpha.
15. A method of reducing inflammatory cytokine response in an animal in need thereof, comprising:

administering to said animal an immune modulating amount of a plant or plant product that includes a Coronavirus spike protein.

16. The method of claim 15 wherein said S protein is fused to another protein.
17. The method of claim 15 wherein said plant or plant produce is administered as an immunological modulating booster composition in combination with a vaccine.
18. The method of claim 15 wherein said Spike protein is produced by or administered as a part of a plant.
19. The method of claim 15 wherein said booster composition is administered orally.
20. The method of claim 15 wherein said Spike protein booster composition is administered at a level that will not induce antibody protection.
21. The method of claim 17 wherein said Spike protein booster composition reduces the inflammatory cytokine response in said animal by altering cytokine levels.
22. The method of claim 15 wherein said cytokines are one or more of GM-CSF, IFN gamma, IL-lalpha, IL-1beta, IL-1ra, IL-2, IL-4, IL-6, IL-8, IL-10, IL-12, IL18, or TNF alpha are altered after administration of the S protein.
23. The method of claim 22 wherein said cytokine level is one or more of GN-CSF, IFN gamma, and/or TNF alpha.
24 An immune modulating composition that decreases an inflammatory cytokine response comprising:

a plant produced or plant or plant product which has been modified to expresses a Coronavirus S-protein.

25. The composition of claim 24 wherein said S-protein is produced in and/or is present as a part of plant material.
26. The composition of claim 24 wherein said S-Protein is from PEDV.
27. The composition of claim 24 wherein said S protein is fused to another protein.
28. The composition of claim 26 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that the inflammatory cytokine response is reduced prior to infection.
29. A method of immunologically protecting nursing newborn animals comprising;
administering a plant or plant product comprising a Coronavirus S protein to the mother so that passive immunity is transferred from the mother's milk to the newborn animals.
30. The method of claim 29 wherein said animals is a sow/gilt.
31. The method of claim 29 wherein said Coronavirus introduction is via oral administration
32. The method of claim 29 wherein said Coronavirus is PEDV.
33. The method of claim 29 where the S protein is fused to another protein.
34. The method of claim 32 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that inflammatory cytokine levels are reduced prior to infection.
35. A method of reducing cytokine induced inflammatory response in nursing animals comprising;
administering a plant or plant product comprising a Coronavirus S protein to the mother so that passive immunity is transferred from the mother's milk to the newborn animals.
36. The method of claim 35 wherein said animals is a sow/gilt.
37. The method of claim 35 wherein said animals is a sow/gilt.
38. The method of claim 35 wherein said Coronavirus introduction is via oral administration
39. The method of claim 35 wherein said Coronavirus is PEDV.
40. The method of claim 35 where the S protein is fused to another protein.
41. The method of claim 39 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that inflammatory cytokine levels are reduced prior to infection.

### EXAMPLES

### Example 1

The spike protein is the primary immunogen due to its many neutralizing epitopes and a likely vaccine candidate. A number of prototype candidates based on different portions of the spike protein have shown promising immune responses in animal studies (Oh et al., 2014; Makadiya et al., 2016). These include immunogens based on the S1 moiety (Oh et al., 2014) (Makadiya et al., 2016), the S2 moiety (Okda et al., 2017), and a smaller portion known as the core neutralizing epitope or COE (amino acids 499-638) that has been identified as containing neutralizing epitopes (Chang et al., 2002). However, the prototype vaccines require the purification of the S protein which has been difficult to produce at high levels in several recombinant systems (Makadiya et al., 2016; Piao et al., 2016) (Van Noi and Chung, 2017). In addition, parenterally delivered vaccines are labor-intensive for commercial operations and not likely to provide the strong mucosal response thought to be required for better protection against transmission across the mucus membranes.

Oral administration would eliminate the need for injection and greatly facilitate widespread vaccination against PEDV. Precedent for oral immunization for PEDV includes a number of studies expressing PEDV S or N proteins in probiotics such as Lactobacillus. Oral delivery of these products elicits an immune response and protection upon challenge (Di-qiu et al., 2012; Hou et al., 2018). The S protein for PEDV has been produced in tobacco, rice and other plants and elicits an immune response with neutralizing activity against the virus (Kang et al., 2005); (Bae et al., 2003; Huy et al., 2012; Huy and Kim, 2019). Ideally this protection could be achieved in a system in which the antigen is stable during production, storage and transport and does not require purification of the antigen away from other toxic compounds.

Maize grain has emerged as a preferred option for oral vaccines as it provides high levels of accumulation of the recombinant proteins and bioencapsulation of the protein to protect it from degradation the digestive tract. The maize system also has many inherent properties making it amenable to create a practical low-cost oral vaccine for livestock such as; stability of recombinant proteins that retain activity for years in the grain allowing for long-term storage, transport at ambient temperatures, processing of the grain at will rather than a requirement to process large batches immediately upon harvest, and as it is a major component of feed, it provides a safe and non-diluted matrix for delivery.

Early studies with a spike protein from another coronavirus, porcine transmissible gastroenteritis virus (TGEV), demonstrated that an orally-delivered maize-based candidate vaccine elicited an immune response and provided protection upon a challenge in pigs. Other maize-based vaccines have also shown efficacy in animal trials (Hayden et al., 2015) and safety in a human clinical trial. There is also one report of expression of the PEDV spike protein COE in maize, which elicited an immune response in mice (Man et al., 2014). Our previous work demonstrated high expression of the S antigen in maize that would allow for a heat stable, low-cost production supply of the antigen. In addition, when pigs were oral administered the maize-produced S protein, high levels of sera neutralization antibodies were observed after a challenge with the virus. However, because the disease symptoms are acute only in nursing pigs, protection from the virus could not be determined. In this report, we administered the vaccine candidate to naïve sow/gilts and after farrowing, their newborn pigs were challenged with the virus to determine if the dams could provide passive immunization to the newborn pigs.

### Materials and Methods

### Production of Maize Produced-S Antigen. Example 1

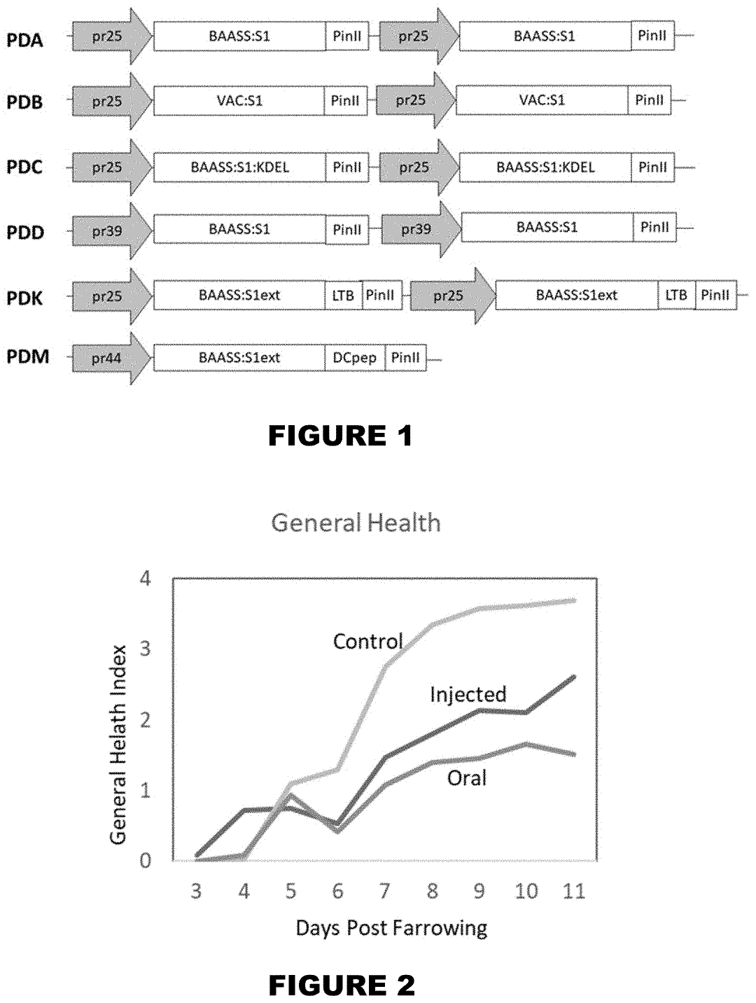

The Spike (S1) nucleotide sequence was introduced into constructs as outlined below and in FIG. 1. S1 refers to the 2154 bp nucleotide sequence set forth in FIG. 1. S1(ext) refers to a 2307 bp sequence (SEQ ID NO: 1). The protein encoded by the sequences is SEQ ID NO: 2. BAASS refers to the barley alpha amylase signal sequence (SEQ ID NO: 3, the polypeptide encoded is SEQ ID NO: 4), Pinll refers to the potato proteinase inhibitor polyadenylation sequence, M refers to a PEDV matrix protein encoding nucleotide sequence (SEQ ID NO: 7, the polypeptide encoded is SEQ ID NO: 8), N refers to PEDV N protein (a SEQ ID NO: 10, the polypeptide encoded is SEQ ID NO: 11); DR13 refers to a South Korea strain of the virus (SEQ ID NO: 25 is the nucleotide, the encoded polypeptide is SEQ ID NO: 9); COE refers to a small portion of the S1 protein that is involved in the immune response and the sequence set forth below (SEQ ID NO: 12). DCpep refers to the dendritic binding peptide. An embodiment provides the Spike polypeptide is fused to a dendritic cell targeting sequence, (DC3) (SEQ ID NO: 13), and/or a heat labile enterotoxin B subunit (LtB) peptide (SEQ ID NO: 14 and the polypeptide encoded is SEQ ID NO: 15). Dendritic cells are antigen-presenting cells that participate in activation of T cells. Polypeptides may be targeted to dendritic cells. See Mohamadzadeh et al. (2009) “Dendritic cell targeting of Bacillus anthracis protective antigen expressed by Lactobacillus acidophilus protects mice from lethal challenge” Proc. Natl. Acad. Sci USA 106, 4331-4336.

Reference to the promoter pr25 refers to the maize globulin-1 gene (SEQ ID NO: 16), pr39 refers to a maize 27 kD gamma-zein gene promoter (SEQ ID NO: 17); and pr44 refers to the pr25 globulin-1 promoter, with two extra copies of a portion of the promoter (SEQ ID NO: 18).

All fragments were optimized for maize codon usage and synthesized by Genescript. Full length coding sequence fragments were synthesized for the constructs with the US or DR13 strains with the BAASS signal sequence, as well as for the COE-DC peptide construct. Constructs with vacuolar or KDEL signal sequences (SEQ ID NO: 19) were prepared by synthesis of partial fragments and reconstruction of the full coding region using Ncol, EcoR1 and Hindlll restriction sites to exchange with the BAASS full length S1 synthesized fragments. A partial fragment was also ordered for the S1 Ext and used to reconstruct the full coding region by restriction digestion with Hindlll+PacI. Cloning into the pSB11 vector was by Ncol and PacI restriction sites. Constructs with double copies of the complete promoter+coding region transcription unit were prepared by digestion with Ascl and MluI and ligation of the second copy of the transcription unit.

The entire PEDV sequence is SEQ ID NO: 21, with the Spike protein encoded SEQ ID NO: 22.

The S protein is extremely difficult to express in other hosts and therefore we did not know what to expect in the maize grain. It was surprising that the construct PDA with an apoplast targeting sequence provided poor expression levels of the protein while the PDC construct with an ER targeting sequence demonstrated good levels of expression Transgenic maize containing the S protein (PDC) as previously described was grown to obtain grain that was used for the study. The grain was enriched for germ using our customized germ fractionation equipment and the processed grain dried to a moisture content of less than 12% and then put through the Glen Mill grinder according to obtain a course corn meal such that >80% of the material could pass through a 20-mesh screen.

Western blot analysis. Samples of the grain were extracted and Western gels performed to determine the levels of S protein. Proteins were extracted from ground seed with 1×PBS+1% SDS, loaded onto a 4-12% bis-tris gel (LifeTech), and transferred to PDVF membrane by iBlot. The blot was incubated in Pacific Immunology's custom rabbit anti-PEDV S1 overnight at a dilution of 1:2000 and developed with anti-rabbit-alkaline phosphatase conjugate at a dilution of 1:2000 (Jackson Immunoresearch #111-055-003) and BCIP-NBT liquid substrate (Sigma #B1911). Ten ng of the COE standard synthesized by Genescript was loaded as a positive control and the concertation of S1 was estimated visually using this standard

Preparation of material for animal trials. The corn meal was placed in individual bags (1 kg) that was given to the animals in one day. Non-transgenic commodity corn was used for the control and to blended when needed to give the predetermined dose. The dose was calculated to contain 10 mg of the S antigen using the COE peptide as the standard. Bags were labeled with letter and color codes and given to those conducting the animals trial but without the key as to what each treatment consisted of.

Animals. PIC 1050 dams were bred with semen from PIC 337 boars (both are white crosses). PIC 1050 is a large white/Yorkshire commercial crossbred and the PIC 337 is a commercial Yorkshire crossbred boar/semen. All sows/gilts used in this study were determined to be free from PEDV. Prior to delivery to the test facility, all pigs received a dose of PCV2 vaccine (Circoflex®), antibiotic (0.3mLs Excede®) and a dose of Vitamin E (Vital E®). All pigs were housed in a hepafiltered isolation room performed under BL-2 conditions. Each animal was given two ear tags to uniquely identify the animal. Animals were weighed and assigned a random number (with Excel random number generator) and then sorted by projected farrow dates. The dams were then assigned a specific group by ascending order and assigned to a treatment group in the farrow blocks. Pens were raised plastic tube (4 ft×5 ft) with plastic slatted flooring.

### Animal Trial

Four pigs per pen (five pens total) were used. Feed was a commercially available type that was appropriate for age/size of pigs (Purina® brand). Feed was provided ad libitum via a six-hole plastic nursery feeder. Water was provided ad libitum via one nipple waterer. Water was sourced from the on-site rural well. Photoperiod was controlled by a timer and provided 15 hours of light and 9 hours of dark. Room temperature was monitored daily with a high/low thermometer (range of 68°-83° F.). Each treatment group consisted of 4 animals. Animals were separated by treatment group (4 animals/treatment group). The treatment groups consisted of the following shown in Table 1.

TABLE 1
Treatment Group

| Group | Primary | Boost 1 | Boost 2 | Condition |
|---|---|---|---|---|
| 1 | Inject | Inject | Inject | Parenteral delivery-positive control |
| 2 | Oral | Oral | Oral | No vaccine-negative control |
| 3 | PDC Oral 1× | PDC Oral 1× | PDC Oral 1× | Oral prime and boost 1× |

All pigs were observed daily for general health. Fecal matter, blood and milk samples were taken and frozen until analysis was performed.

### Treatments

One treatment consisted of pigs that were administered intramuscularly with a commercially available killed virus vaccine (Zoetis). A second treatment consisted of providing oral doses the material as described above (1 kg of maize meal) for three consecutive days for each dose. Three doses were administered; six days after acclimation, 1 month after the primary dose and, 10 days pre-farrowing. The third group was administered untransformed ground maize germ following the same schedule. The animals were fasted for four hours before being offered the maize material and returned to their normal diet an hour after administration. Each animal was hand fed and consumed the full dose of maize material offered.

At farrowing, each sow was placed in a separate pen with its piglets and each treatment group was housed in separate rooms. All animals were observed daily for changes in general health. Fecal, serum, and milk samples were taken and stored frozen on the days indicated in Table 2. When administration of the candidates and sample collection fell on the same day, all samples were taken prior to administration of injected or oral material. Blood was collected, clotted and sera centrifuged and placed in vials.

### Challenge

Upon farrowing, each sow was kept separately with its newborn pigs for the remainder of the study. Daily observations with special attention to diarrhea symptoms and weights were taken every 2-3 days. All piglets were weighed at day of farrowing (DOF) and on days 3, 6, 9 and 11 days post challenge (DPC). Sows with high litter numbers were culled such that no sow had more than 14 piglets for the remainder of the study. Virus for the challenge was obtained from Dr. Jianqiang Zhang at Iowa State University was obtained (ISU batch #PEDV USA/NC/49469/2013 at the titer of 10\hat{ }3 TCID50/ml). Virus (10 ml) was given 3-5 days after farrowing to each piglet by IG to make sure they got a full dose.

### Diagnostics

Viral titers were assessed to detect the virus on sows prior to the start of the experiment to confirm they are free of the virus using PCR (PEDV/PDCoV/TGEC Multiplex and the NAB assay (PEDV HTNT assay) at ISU Veterinary Diagnostic Laboratory. Serum samples were collected on days of administration and 2 weeks following. Milk samples were collected on the day of farrowing as well as several time points afterwards. Neutralization assays for sera and milk samples were performed by the Veterinary Diagnostic Laboratory at South Dakota State University.

### Statistics

A mixed effects analysis of variance was used to analyze the data with the final titer as response variable of interest and the treatment group and the weight group as factors. Weight group was included in the analysis as a random effect to account for any variation in the titers due to animal weight. Differences in the treatment groups were compared using Tukey's HSD procedure with a 5% significance level. The data were analyzed on a base-2 log scale. Comparing the titers on the log scale detects significant differences between the treatment groups (p-value=0.05). The differences between groups were determined using the Tukey HSD method.

### Results

### Virus Challenge on Nursing Piglets.

The mean weight of the piglets per treatment group on the day of challenge (DOC) is shown in Table 1. The slightly higher weight of piglets in Treatment 1 is most likely due to the lower number in the litter compared to the other groups (˜9 versus ˜12).

TABLE 2
Mean weight of piglets in each group
of the test. Group 1 showed a slightly higher
weight than the other groups likely due to a

| Treatment | Mean Weight |
|---|---|
| 1 | 4.242 |
| 2 | 3.281 |
| 3 | 3.663 |

Starting on the day of challenge (DOC) each pig was observed daily for dehydration, diarrhea, and general health. The observations were recorded using the key in Table 3 These criteria were then used to calculate a General Health Index by adding the scores and the results shown in FIG. 2.

TABLE 3
Observation Key

| | | General |
|---|---|---|
| Diarrhea | Dehydration | Health |
| 0 = none | 0 = none | 0 = Normal |
| 1 = Loose | 1 = Mild, spine | 1 = Lethargic |
| and pasty | prominent | 2 = Vomiting |
| 2 = Watery | 2 = Sever, spine is | 3 = Anorexic |
| | prominent, rib cage | 5 = Dead |
| | and waist observed | |
| | when viewed from | |
| | above, abdomen is | |
| | tucked up when | |
| | viewed from the side | |

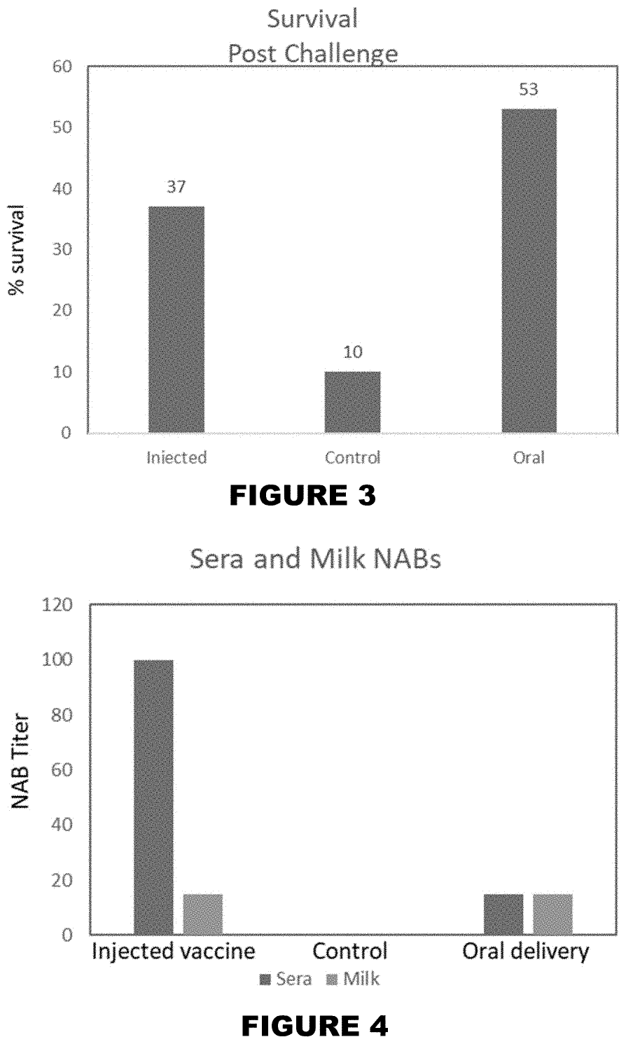

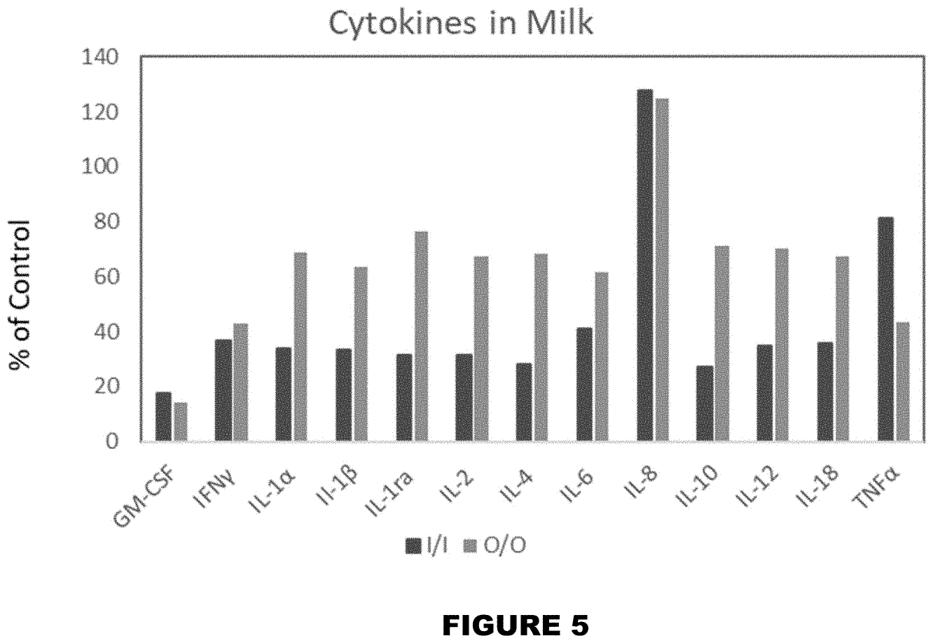

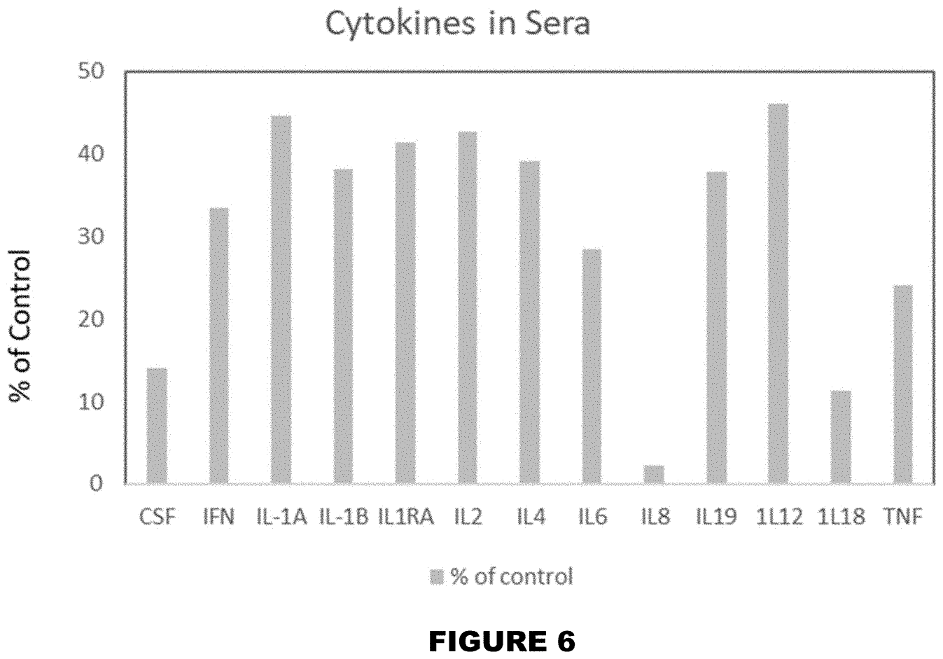

There was a high mortality rate overall for the piglets nursing on the control sows resulting in the piglet survival being the most meaningful measurement as shown in FIG. 3. The results show that the virus level selected was very effective in that >90% of the piglets that nursed on dams not vaccinated died following challenge. In contrast, 53% of the piglets survived the challenge when nursed on dams administered the oral vaccine candidate. The parenteral commercial vaccine provided an intermediate level of protection (37% survival).

The data was statistically analyzed using a logistic mixed model analysis with alive/dead as the response, treatment and challenge weight as predictors, and sow as a random effect. The analysis was done primarily using the glmer command of the lme4 package in R version 4.0.4. Other packages such as multcomp and emmeans were used to further analyze the model estimates. Tukey's HSD procedure at 90% overall confidence was used to separate the treatments.

The weight is known to influence the severity of intestinal disease and therefore was taken into account. In particular, pigs in Treatment 1 piglets were significantly heavier than average and the model adjusts for the Day 0 weight, which was a highly significant predictor of survival. The model adjusts the survival rates of each treatment as if they all had the same average weight. So, part of the higher survival rate for Treatment 1 is being attributed (appropriately) to the weight effect rather than the treatment effect. Once the weight effect is accounted for, the effect of Treatment 1 is lower. This was confirmed this by running a model without the Day 0 weight. The values in Table 4 results were verified using 3 different computer programs (R, SAS, and SPSS) to make sure that there wasn't an error in the R package initially used for the analysis. They all match up with only tiny differences due to the different estimation algorithms used by the programs. The different letters indicate significant differences between treatments.

TABLE 4
Statistical Probability of Survival

| Treatment | Probability | Letters |
|---|---|---|
| 1 | 0.2227 | ab |
| 2 | 0.0647 | a |
| 3 | 0.5505 | b |

In summary, the weight of the piglets at Day 0 and the treatment group are significant predictors of survival to the end of the experiment (p<0.0001 and p=0.0036). The oral treatment group was the only group that had a significantly higher survival rate than the controls with 90% overall confidence.

### Correlates of Protection

The ability to correlate specific biomarkers with protection to the pathogen has proven to be a useful approach to help in understanding the mechanism of action. Therefore, we explored this by examining sera, colostrum and milk samples from the sows and tried to correlate this with protection.

FIG. 4 illustrates the mean values for milk and sera NABs of the different groups. The figure shows that sows that received the injected vaccine had the highest titers for sera NABs. Lower levels of NABs were detected in the sera of the orally administered group. Both the injected and oral groups had lower levels of NABs in the milk while the control group was negative for both sera and milk.

In addition to the NABs, cytokine levels were analyzed in the various treatments. The samples were analyzed by Eve Technologies which reported values in pg/ml of cytokines for 13 different cytokines and the mean value calculated. The mean value was then used to normalize the results to the control and the percent of control shown is shown below in FIG. 4 for the milk and 5 for sera.

### Discussion

Newborn piglets are most susceptible to PEDV and this study demonstrated that after the viral challenge there was a 90% mortality in the control group. The orally delivered treatment provided protection from the virus demonstrating that this can be an effective manner to deliver efficacious vaccines to swine. NABs could be detected in both sow sera and milk that may be in part responsible for providing protection. However, unexpectedly sows that received the S protein either parenterally or orally had a reduced level of most cytokines. This reduced level of cytokines is passed on through the milk to the nursing piglets. As coronavirus is known to create a “cytokine storm” in which the high levels of cytokines are thought to be associated with the most severe symptoms leading to death. This reduced level of most cytokines may play a significant role in protection (consistent with this trend, INF levels increased, but this is desirable for a reduced non-specific reaction). This phenomenon is likely to occur for other S proteins from other coronaviruses as well. Furthermore, the reduction in cytokine levels may help protect animals from other diseases where cytokine levels play a significant role.

### REFERENCES

Bae, J.-L., J.-G. Lee, T.-J. Kang, H.-S. Jang, Y.-S. Jang, and M.-S. Yang. 2003. Induction of antigen-specific systemic and mucosal immune responses by feeding animals transgenic plants expressing the antigen. Vaccine 21(25):4052-4058.
Chang, S.-H., J.-L. Bae, T.-J. Kang, J. Kim, G.-H. Chung, C.-W. Lim, H. Laude, M.-S. Yang, and Y.-S. Jang. 2002. Identification of the epitope region capable of inducing neutralizing antibodies against the porcine epidemic diarrhea virus. Molecules and cells 14(2):295-299.
Chang, Y.-C., C.-Y. Chang, P.-S. Tsai, H.-Y. Chiou, C.-R. Jeng, V. F. Pang, and H.-W. Chang. 2018. Efficacy of heat-labile enterotoxin B subunit-adjuvanted parenteral porcine epidemic diarrhea virus trimeric spike subunit vaccine in piglets. Applied microbiology and biotechnology 102(17):7499-7507.
Di-qiu, L., G. Jun-wei, Q. Xin-yuan, J. Yan-ping, L. Song-mei, and L. Yi-jing. 2012. High-level mucosal and systemic immune responses induced by oral administration with Lactobacillus-expressed porcine epidemic diarrhea virus (PEDV) S1 region combined with Lactobacillus-expressed N protein. Applied microbiology and biotechnology 93(6):2437-2446.
Gerdts, V., and A. Zakhartchouk. 2017. Vaccines for porcine epidemic diarrhea virus and other swine coronaviruses. Veterinary microbiology 206:45-51.
Guo, J., L. Fang, X. Ye, J. Chen, S. Xu, X. Zhu, Y. Miao, D. Wang, and S. Xiao. 2019. Evolutionary and genotypic analyses of global porcine epidemic diarrhea virus strains. Transboundary and emerging diseases 66(1):111-118.
Hayden, C. A., E. M. Egelkrout, A. M. Moscoso, C. Enrique, T. K. Keener, R. Jimenez-Flores, J. C. Wong, and J. A. Howard. 2012. Production of highly concentrated, heat-stable hepatitis B surface antigen in maize. Plant Biotechnology Journal 10(8):979-984. doi: DOI 10.1111/j.1467-7652.2012.00727.x
Hayden, C. A., M. E. Fischer, B. L. Andrews, H. C. Chilton, D. D. Turner, J. H. Walker, I. R. Tizard, and J. A. Howard. 2015. Oral delivery of wafers made from HBsAg-expressing maize germ induces long-term immunological systemic and mucosal responses. Vaccine 33(25):2881-2886.
Hayden, C. A., E. M. Smith, D. D. Turner, T. K. Keener, J. C. Wong, J. H. Walker, I. R. Tizard, R. Jimenez-Flores, and J. A. Howard. 2014. Supercritical fluid extraction provides an enhancement to the immune response for orally-delivered hepatitis B surface antigen. Vaccine 32(11):1240-1246.
Hood, E. E., S. P. Devaiah, G. Fake, E. Egelkrout, K. Teoh, D. V. Requesens, C. Hayden, K. R. Hood, K. M. Pappu, J. Carroll, and J. A. Howard. 2012. Manipulating corn germplasm to increase recombinant protein accumulation. Plant Biotechnology Journal 10(1):20-30. doi: DOI 10.1111/j.1467-7652.2011.00627.x
Hood, E. E., G. L. Helmer, R. T. Fraley, and M. D. Chilton. 1986. The hypervirulence of Agrobacterium tumefaciens A281 is encoded in a region of pTiBo542 outside of T-DNA. J Bacteriol 168(3):1291-1301.
Hou, X., X. Jiang, Y. Jiang, L. Tang, Y. Xu, X. Qiao, M. Liu, W. Cui, G. Ma, and Y. Li. 2018. Oral Immunization against PEDV with Recombinant Lactobacillus casei Expressing Dendritic Cell-Targeting Peptide Fusing COE Protein of PEDV in Piglets. Viruses 10(3):106.
Huy, N.-X., and M.-Y. Kim. 2019. Improved expression of porcine epidemic diarrhea antigen by fusion with cholera toxin B subunit and chloroplast transformation in Nicotiana tabacum. Plant Cell, Tissue and Organ Culture (PCTOC):1-11.
Huy, N.-X., S.-H. Kim, M.-S. Yang, and T.-G. Kim. 2012. Immunogenicity of a neutralizing epitope from porcine epidemic diarrhea virus: M cell targeting ligand fusion protein expressed in transgenic rice calli. Plant cell reports 31(10):1933-1942.
Ishida, Y., H. Saito, S. Ohta, Y. Hiei, T. Komari, and T. Kumashiro. 1996. High efficiency transformation of maize (Zea mays L.) mediated by Agrobacterium tumefaciens. Nat Biotechnol 14(6):745-750. doi: 10.1038/nbt0696-745
Komari, T., Y. Hiei, Y. Saito, N. Murai, and T. Kumashiro. 1996. Vectors carrying two separate T-DNAs for co-transformation of higher plants mediated by Agrobacterium tumefaciens and segregation of transformants free from selection markers. Plant Journal 10(1):165-174.
Lamphear, B. J., J. M. Jilka, L. Kesl, M. Welter, J. A. Howard, and S. J. Streatfield. 2004. A corn-based delivery system for animal vaccines: an oral transmissible gastroenteritis virus vaccine boosts lactogenic immunity in swine. Vaccine 22(19):2420-2424.
Makadiya, N., R. Brownlie, J. van den Hurk, N. Berube, B. Allan, V. Gerdts, and A. Zakhartchouk. 2016. S1 domain of the porcine epidemic diarrhea virus spike protein as a vaccine antigen. Virology journal 13(1):1.
Man, K., B. Yang, J. Xue, X. Qin, H. Zhou, F. Xu, C. Xu, F. Yang, L. Zhang, and X. Li. 2014. Expression of core neutralizing epitope gene of porcine epidemic diarrhea virus in maize. Journal of Agricultural Science and Technology (Beijing) 16(6):28-35.
Oh, J., K.-W. Lee, H.-W. Choi, and C. Lee. 2014. Immunogenicity and protective efficacy of recombinant S1 domain of the porcine epidemic diarrhea virus spike protein. Archives of virology 159(11):2977-2987.
Piao, D.-C., Y.-S. Lee, J.-D. Bok, C.-S. Cho, Z.-S. Hong, S.-K. Kang, and Y.-J. Choi. 2016. Production of soluble truncated spike protein of porcine epidemic diarrhea virus from inclusion bodies of Escherichia coli through refolding. Protein expression and purification 126:77-83.
Streatfield, S. J., J. M. Mayor, D. K. Barker, C. Brooks, B. J. Lamphear, S. L. Woodard, K. K. Beifuss, D. V. Vicuna, L. A. Massey, and M. E. Horn. 2002. Development of an edible subunit vaccine in corn against enterotoxigenic strains of Escherichia coli. In Vitro Cellular & Developmental Biology-Plant 38(1):11-17.
Sun, D., L. Feng, H. Shi, J. Chen, X. Cui, H. Chen, S. Liu, Y. Tong, Y. Wang, and G. Tong. 2008. Identification of two novel B cell epitopes on porcine epidemic diarrhea virus spike protein. Veterinary microbiology 131(1-2):73-81.
Van Noi, N., and Y.-C. Chung. 2017. Optimization of expression and purification of recombinant S1 domain of the porcine epidemic diarrhea virus spike (PEDV-S1) protein in Escherichia coli. Biotechnology & Biotechnological Equipment 31(3):619-629.
Wang, X., L. Wang, X. Huang, S. Ma, M. Yu, W. Shi, X. Qiao, L. Tang, Y. Xu, and Y. Li. 2017. Oral delivery of probiotics expressing dendritic cell-targeting peptide fused with porcine epidemic diarrhea virus COE antigen: a promising vaccine strategy against PEDV. Viruses 9(11):312.
Chang, S.-H., J.-L. Bae, T.-J. Kang, J. Kim, G.-H. Chung, C.-W. Lim, H. Laude, M.-S. Yang, and Y.-S. Jang. 2002. Identification of the epitope region capable of inducing neutralizing antibodies against the porcine epidemic diarrhea virus. Molecules and cells 14(2):295-299.
Choe, S., S. Song, D. Piao, G.-N. Park, J. Shin, Y.-J. Choi, S.-K. Kang, R. M. Cha, B.-H. Hyun, and B.-K. Park. 2020. Efficacy of orally administered porcine epidemic diarrhea vaccine-loaded hydroxypropyl methylcellulose phthalate microspheres and RANKL-secreting L. lactis. Veterinary Microbiology 242:108604.
Gerdts, V., and A. Zakhartchouk. 2017. Vaccines for porcine epidemic diarrhea virus and other swine coronaviruses. Veterinary microbiology 206:45-51.
Hood, E. E., S. P. Devaiah, G. Fake, E. Egelkrout, K. Teoh, D. V. Requesens, C. Hayden, K. R. Hood, K. M. Pappu, J. Carroll, and J. A. Howard. 2012. Manipulating corn germplasm to increase recombinant protein accumulation. Plant Biotechnology Journal 10(1):20-30. doi: DOI 10.1111/j.1467-7652.2011.00627.x
Kang, T.-J., Y.-S. Kim, Y.-S. Jang, and M.-S. Yang. 2005. Expression of the synthetic neutralizing epitope gene of porcine epidemic diarrhea virus in tobacco plants without nicotine. Vaccine 23(17):2294-2297.
Makadiya, N., R. Brownlie, J. van den Hurk, N. Berube, B. Allan, V. Gerdts, and A. Zakhartchouk. 2016. S1 domain of the porcine epidemic diarrhea virus spike protein as a vaccine antigen. Virology journal 13(1):1.
Oh, J., K.-W. Lee, H.-W. Choi, and C. Lee. 2014. Immunogenicity and protective efficacy of recombinant S1 domain of the porcine epidemic diarrhea virus spike protein. Archives of virology 159(11):2977-2987.
Okda, F. A., S. Lawson, A. Singrey, J. Nelson, K. S. Hain, L. R. Joshi, J. Christopher-Hennings, E. A. Nelson, and D. G. Diel. 2017. The S2 glycoprotein subunit of porcine epidemic diarrhea virus contains immunodominant neutralizing epitopes. Virology 509:185-194.

### Example 2

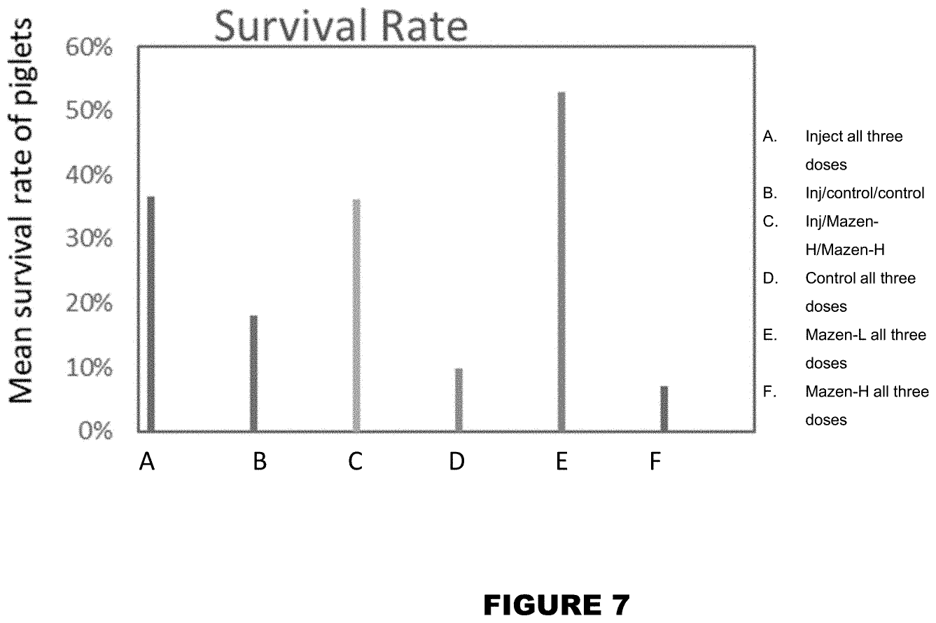

Six groups that were tested. FIG. 7 shows the mean survival rates of the different groups. The key is to the right. The booster dose is C where the primary dose was the commercial vaccine and the boosts were our oral candidate. Group B is when there was no boost after the primary dose. The results suggest that the oral boosting was as effective as boosting by injection.

Group F (HO) refers to the high oral dose (5× the low dose). In this case the survival rate is at control levels.

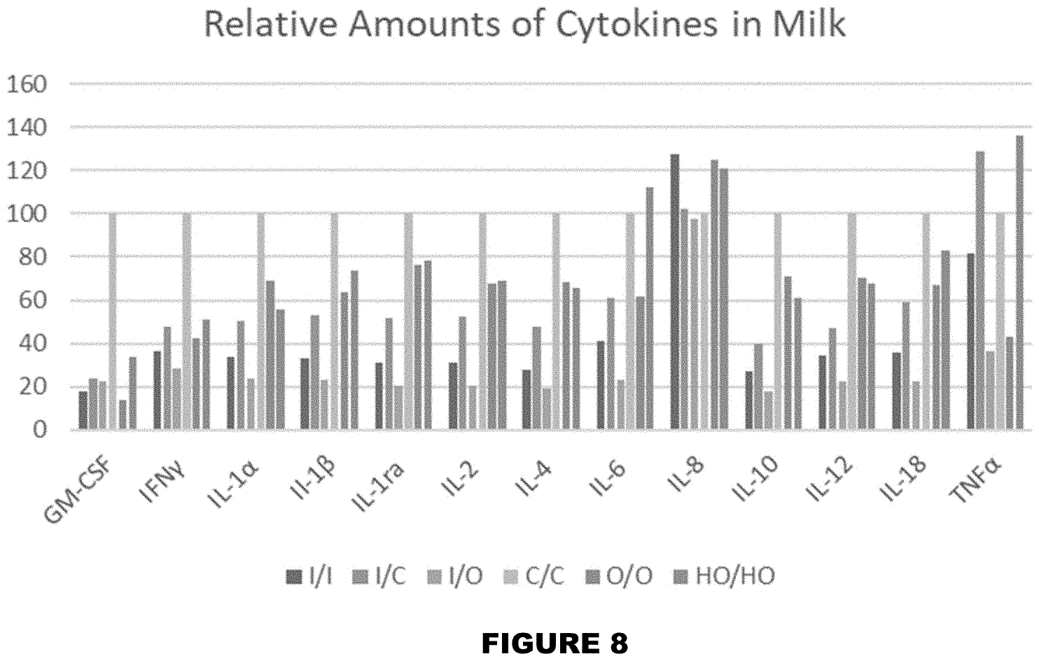

FIG. 8 shows the levels of cytokines in milk for all six treatment groups. Of special interest is group F as it shows higher levels of TNF and compared to those in group D our low oral dose oral treatment.

### Example 3

In a different study, three groups of 5 sows each were dosed either Control, High Dose PEDV vaccine or Low Dose PEDV vaccine. Three doses were administered at 9 weeks, 5 weeks and 2 weeks pre-farrowing. Three cytokines (IFNγ, GM-CSF, and TNFα were measured at day of farrowing, day of challenge (DO Post Challenge) and day 3 post challenge (D3 Post challenge).

The tables below show data generated for different cytokines.

TABLE 5
Milk cytokines by treatment group-Linear Model All Litters Included

| | Treatment Group | | | | |
|---|---|---|---|---|---|
| | Control | High Dose | Low Dose | SE | P-value |
| Sample size, n | 5 | 5 | 5 | | |
| Average BW @ | 4.40 | 3.83 | 4.34 | 0.23 | 0.19 |
| challenge · lb | | | | | |
| Piglet survivability, | 44.5 | 50.0 | 57.0 | 0.13 | 0.79 |
| % | | | | | |
| IFNγ, ng/mL | | | | | |
| Day of farrowing | 39.8 | 42.0 | 63.5 | 11.8 | 0.33 |
| D0 Post challenge | 19.3 | 16.8 | 62.7 | 15.5 | 0.10 |
| D3 Post challenge | 20.6 | 22.1 | 63.6 | 19.1 | 0.24 |
| GM-CSF, ng/mL | | | | | |
| Day of farrowing | 1.88^{x} | 0.68^{y} | 1.86^{x} | 0.37 | 0.06 |
| D0 Post challenge | 0.36^{a,b} | 0.00^{a} | 1.02^{b} | 0.26 | 0.05 |
| D3 Post challenge | 0.33^{x,y} | 0.11^{x} | 1.29^{y} | 0.53 | 0.10 |
| TNFα, ng/mL | | | | | |
| Day of farrowing | 1.17^{a} | 0.36^{b} | 1.09^{a,b} | 0.21 | 0.03 |
| D0 Post challenge | 0.372^{a,b} | 0.00^{a} | 1.07^{b} | 0.29 | 0.06 |
| D3 Post challenge | 0.38 | 0.02 | 1.16 | 0.36 | 0.12 |

^{a,b}Means within a row with different letters are different (P ≤ 0.05);
^{x,y}Means within a row with different letters are different (P ≤ 0.10).

TABLE 6
Milk cytokines by treatment group-Linear Model-Outliers removed

| | Treatment Group | | | | |
|---|---|---|---|---|---|
| | Control | High Dose | Low Dose | SE | P-value |
| Sample size, n | 4 | 4 | 4 | | |
| Average BW @ | 4.35 | 4.03 | 4.19 | 0.31 | 0.62 |
| challenge · lb | | | | | |
| Piglet survivability, | 36.2^{x} | 62.5^{x,y} | 66.7^{y} | 9.3 | 0.09 |
| % | | | | | |
| IFNγ, ng/mL | | | | | |
| Day of farrowing | 35.8 | 30.6 | 63.2 | 19.1 | 0.19 |
| D0 Post challenge | 6.04^{a} | 3.92^{a} | 78.34^{b} | 8.7 | 0.0003 |
| D3 Post challenge | 2.24^{a} | 7.41^{a} | 79.50^{b} | 18.1 | 0.003 |
| GM-CSF, ng/mL | | | | | |
| Day of farrowing | 1.83 | 0.71 | 2.10 | 0.44 | 0.11 |
| D0 Post challenge | 0.13^{a} | 0.00^{a} | 1.26^{b} | 0.23 | 0.006 |
| D3 Post challenge | 0.13^{x} | 0.14^{x} | 1.62^{y} | 0.40 | 0.04 |
| TNFα, ng/mL | | | | | |
| Day of farrowing | 1.02 | 0.39 | 1.15 | 0.24 | 0.11 |
| D0 Post challenge | 0.02^{a} | 0.00^{a} | 1.31^{b} | 0.19 | 0.001 |
| D3 Post challenge | 0.00^{a} | 0.03^{a} | 1.46^{b} | 0.30 | 0.01 |

^{a,b}Means within a row with different letters are different (P ≤ 0.05);
^{x,y}Means within a row with different letters are different (P ≤ 0.10).

What is claimed is:

1. A method of providing passive immunity protection to an animal from the effects of Coronavirus introduction, comprising:
administering to said animal prior to farrowing a composition of a plant or plant product comprising the Spike (S1) protein of Coronavirus.

2. The method of claim 1, wherein said Coronavirus introduction is via oral administration

3. The method of claim 1 wherein said Coronavirus is PEDV.

4. The method of claim 1 where the S protein is fused to another protein.

5. The method of claim 3 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that inflammatory cytokine levels are altered to reduce inflammation prior to infection.

6. The method of claim 1 wherein said animal is a dam.

7. The method of claim 1 wherein said administration includes a booster prior to farrowing.

8. The method of claim 6 wherein said dam passes protection to her piglets.

9. The method of claim 1 wherein is said administration is 3 doses.

10. The method of claim 1 wherein said plant or plant product is seed which expresses said S Protein.

11. The method of claim 8 wherein said seed is dosed with animal feed.

12. The method of claim 1 wherein said composition decreases cytokine inflammatory response by altering cytokine levels in said animal.

13. The method of claim 1 wherein said cytokine level that is altered includes one or more of GM-CSF, IFN gamma, IL-lalpha, IL-1beta, IL-1ra, IL-2, IL-4, IL-6, IL-8, IL-10, IL-12, IL18, or TNF alpha.

14. The method of claim 13 wherein said cytokine level is one or more of GN-CSF, IFN gamma, and/or TNF alpha.

15. A method of reducing inflammatory cytokine response in an animal in need thereof, comprising:
administering to said animal an immune modulating amount of a plant or plant product that includes a Coronavirus spike protein.

16. The method of claim 15 wherein said S protein is fused to another protein.

17. The method of claim 15 wherein said plant or plant produce is administered as an immunological modulating booster composition in combination with a vaccine.

18. The method of claim 15 wherein said Spike protein is produced by or administered as a part of a plant.

19. The method of claim 15 wherein said booster composition is administered orally.

20. The method of claim 15 wherein said Spike protein booster composition is administered at a level that will not induce antibody protection.

21. The method of claim 17 wherein said Spike protein booster composition reduces the inflammatory cytokine response in said animal by altering cytokine levels.

22. The method of claim 15 wherein said cytokines are one or more of GM-CSF, IFN gamma, IL-lalpha, IL-1beta, IL-1ra, IL-2, IL-4, IL-6, IL-8, IL-10, IL-12, IL18, or TNF alpha are altered after administration of the S protein.

23. The method of claim 22 wherein said cytokine level is one or more of GN-CSF, IFN gamma, and/or TNF alpha.

24. An immune modulating composition that decreases an inflammatory cytokine response comprising:
a plant produced or plant or plant product which has been modified to expresses a Coronavirus S-protein.

25. The composition of claim 24 wherein said S-protein is produced in and/or is present as a part of plant material.

26. The composition of claim 24 wherein said S-Protein is from PEDV.

27. The composition of claim 24 wherein said S protein is fused to another protein.

28. The composition of claim 26 wherein said Coronavirus PEDV protein comprises SEQ ID NO: 3, 4, 9, 21 or 22 or a sequence having at least 90% identity to SEQ ID NO: 3, 4, 9, 21 or 22 or a functional fragment said S1 protein expressed at levels of at least 10 mg/kg in seed of said plant; so that the inflammatory cytokine response is reduced prior to infection.
